SEQUENCE LISTING (38 sequences)

SEQ ID NO: 1 - DNA, 18 bp, Artificial Sequence
  Primer
caccatcttccaggagcg

SEQ ID NO: 2 - DNA, 19 bp, Artificial Sequence
  Primer
cagtgagcttcccgttcag

SEQ ID NO: 3 - DNA, 23 bp, Artificial Sequence
  Primer
gagcctgatcctgcctctacttg

SEQ ID NO: 4 - DNA, 23 bp, Artificial Sequence
  Primer
cctgcatggcctggtctaagtgc

SEQ ID NO: 5 - DNA, 21 bp, Artificial Sequence
  Primer
gctttgagaccacaccctatg

SEQ ID NO: 6 - DNA, 21 bp, Artificial Sequence
  Primer
ttcaggcaatgctgccagtcc

SEQ ID NO: 7 - DNA, 21 bp, Artificial Sequence
  Primer
ccaaagatgaatgccacagag

SEQ ID NO: 8 - DNA, 20 bp, Artificial Sequence
  Primer
cgaacagcaaatccgagatg

SEQ ID NO: 9 - DNA, 19 bp, Artificial Sequence
  Primer
gctgaagagcgtgactgat

SEQ ID NO: 10 - DNA, 19 bp, Artificial Sequence
  Primer
gaggactgcatgtataatg

SEQ ID NO: 11 - DNA, 18 bp, Artificial Sequence
  Primer
gtgccaattgcctactcc

SEQ ID NO: 12 - DNA, 20 bp, Artificial Sequence
  Primer
ggctcacagacatcacgatc

SEQ ID NO: 13 - DNA, 20 bp, Artificial Sequence
  Primer
ttccagctgccctacaatgg

SEQ ID NO: 14 - DNA, 20 bp, Artificial Sequence
  Primer
gaaggttgtggtggtctccg

SEQ ID NO: 15 - DNA, 19 bp, Artificial Sequence
  Primer
caggaccagagccatcaag

SEQ ID NO: 16 - DNA, 20 bp, Artificial Sequence
  Primer
gatgtcatccagggtggaag

SEQ ID NO: 17 - DNA, 19 bp, Artificial Sequence
  Primer
gctgatctgctctttcttg

SEQ ID NO: 18 - DNA, 20 bp, Artificial Sequence
  Primer
gtgcttggatgacttcttgg

SEQ ID NO: 19 - DNA, 21 bp, Artificial Sequence
  Primer
gtacgatgaggaggcctattc

SEQ ID NO: 20 - DNA, 18 bp, Artificial Sequence
  Primer
cgtgcgatggccacatag

SEQ ID NO: 21 - DNA, 20 bp, Artificial Sequence
  Primer
cgtcatggatgtctacgtgc

SEQ ID NO: 22 - DNA, 19 bp, Artificial Sequence
  Primer
gtagcagaccagcatagtg

SEQ ID NO: 23 - DNA, 21 bp, Artificial Sequence
  Primer
aacagttatgctgtggttgta

SEQ ID NO: 24 - DNA, 21 bp, Artificial Sequence
  Primer
caaacgggatgtattgttacc

SEQ ID NO: 25 - DNA, 24 bp, Artificial Sequence
  Primer
gaacgtcaagtgctagatgcctcg

SEQ ID NO: 26 - DNA, 19 bp, Artificial Sequence
  Primer
gtacacgcagagcagtgcg

SEQ ID NO: 27 - DNA, 18 bp, Artificial Sequence
  Primer
ctgtagagcgagtgttgc

SEQ ID NO: 28 - DNA, 19 bp, Artificial Sequence
  Primer
gtagaggttgacagtgtag

SEQ ID NO: 29 - DNA, 19 bp, Artificial Sequence
  Primer
cgaagcggaaactagagcc

SEQ ID NO: 30 - DNA, 17 bp, Artificial Sequence
  Primer
ccagcttggtcagaagc

SEQ ID NO: 31 - DNA, 20 bp, Artificial Sequence
  Primer
cagctctgtacgatgggcac

SEQ ID NO: 32 - DNA, 20 bp, Artificial Sequence
  Primer
cggttgaaggccttggtagc

SEQ ID NO: 33 - DNA, 18 bp, Artificial Sequence
  Primer
gactatgcagagcctggc

SEQ ID NO: 34 - DNA, 18 bp, Artificial Sequence
  Primer
cttatagctggaggtgcc

SEQ ID NO: 35 - DNA, 20 bp, Artificial Sequence
  Primer
gacgattctgctgaggcctg

SEQ ID NO: 36 - DNA, 18 bp, Artificial Sequence
  Primer
gcccagactaatggtgac

SEQ ID NO: 37 - DNA, 23 bp, Artificial Sequence
  Primer
caaggtcatccatgacaactttg

SEQ ID NO: 38 - DNA, 22 bp, Artificial Sequence
  Primer
gtccaccaccctgttgctgtag

(19) United States
(12) Patent Application Publication
(10) Pub. No.: US 2014/0193424 A1
(43) Pub. Date: Jul. 10, 2014

(54) METHOD AND MEDICAMENT FOR INHIBITING LYMPHANGIOGENESIS

(71) Applicants: Beijing Protgen Ltd., Beijing (CN); Tsinghua University, Beijing (CN)

(72) Inventors: Yongzhang LUO, Beijing (CN); Wei ZHUO, Beijing (CN); Lin JIA, Beijing (CN); Yan FU, Beijing (CN); Guodong CHANG, Beijing (CN)

(21) Appl. No.: 13/962,111

(22) Filed: Aug. 8, 2013

(30) Foreign Application Priority Data
Aug. 9, 2012 (CN) 201210281678.2

(51) Int. Cl. A61K 39/395

(52) U.S. Cl. CPC A61K 39/3955
USPC 424/158.1; 530/389.2; 530/389.6

(57) ABSTRACT

The present invention provides a method for inhibiting lymphangiogenesis in a subject, comprising administering a therapeutically effective amount of a CXCR4 inhibitor and/or a CXCL12 inhibitor to the subject. The invention further provides a method for inhibiting tumor lymphatic metastasis in a cancer patient, comprising administering to the subject (a) a therapeutically effective amount of a CXCR4 inhibitor and/or a CXCL12 inhibitor, in combination with (b) a therapeutically effective amount of a VEGF-C inhibitor and/or a VEGF-D inhibitor and/or a VEGFR-3 inhibitor.

### FIELD OF THE INVENTION

The present invention relates to the field of biopharmaceuticals, in particular, to a method and medicament for inhibiting lymphangiogenesis.

### INCORPORATION BY REFERENCE OF SEQUENCE LISTING

The Sequence Listing in an ASCII text file, named 30339_SEQ.txt of 7 KB, created on Mar. 7, 2014, and submitted to the United States Patent and Trademark Office via EFS-Web, is incorporated herein by reference.

### BACKGROUND OF THE INVENTION

In the body, blood vessels are responsible for delivering oxygen, nutrients and other substances to various tissues, and exchanging substances with surrounding tissues through capillaries. The presence of blood pressure causes plasma to leak continuously from capillaries into interstitial space, which is called interstitial fluid. The main function of lymphatic vessels is collecting and returning such protein-rich fluid to the blood circulation. Water, macromolecules and cells may be absorbed by the lymphatic capillaries located at the blind-ends of lymphatic vessels to form lymph fluid, which is transported through collecting lymphatic vessels and is finally returned to the blood at lymphatic-venous junctions, thereby maintaining body fluid equilibrium. During this process, lymph fluid is filtered in lymph nodes, where foreign substances can be recognized by antigen-presenting cells, eliciting specific immune responses. The lymphatic vessels within the intestinal villi can also absorb dietary fat to form chylomicrons. Lymphatic capillaries are present in skin and most of the internal organs, with the exception of the central nervous system, bone marrow and avascular tissues, such as cartilage, cornea and epidermis^{[1]}.

As far back as in the early 17^{th} century, lymphatic vessels were described. However, due to the lack of specific markers that can distinguish blood vessels from lymphatic vessels, intensive studies on lymphangiogenesis and the functions of lymphatic vessels have been carried out for no more than 20 years. Currently, some lymphatic vessel-specific markers have been discovered, for example: 1) a transcription factor known as Prospero Homeobox Protein 1 (Prox-1), which is crucial for lymphangiogenesis during development and can be used as a marker for lymphatic endothelial cells in human tissues^{[2]}; 2) Podoplanin, a renal glomerular podocyte membrane mucoprotein expressed by lymphatic endothelial cells^{[3]}, which is also required for lymphatic vessel development. Although Podoplanin is also expressed in some non-endothelial cells, it is not expressed in blood vessels so that it can be used as a marker for lymphatic capillaries; 3) Lymphatic Vessel Endothelial Hyaluronan Receptor 1 (LYVE-1), a homologue of CD44 protein, which is expressed on both embryonic and adult lymphatic vessels^{[4]}. Although expressed in liver and splenic sinusoids and macrophages, LYVE-1 is a marker for identifying lymphatic vessels in human and mouse; 4) Vascular Endothelial Growth Factor Receptor-3 (VEGFR-3), a cell surface tyrosine kinase receptor, which represents a signaling pathway for lymphangiogenesis. VEGFR-3 is mainly expressed on adult lymphatic endothelial cells, but it is also expressed on the surface of some blood vessels^{[5]}. VEGFR-3 cannot be used as a marker for lymphatic vessels in a tumor because the expression of VEGFR-3 on the surface of blood vessels in some tumors can be upregulated. The discoveries of these lymphatic vessel-specific markers allow us to identify lymphatic vessels in tissues and to study the regulatory mechanism of lymphatic vessel development in pathological conditions.

In adults, mature lymphatic vessels are usually in a quiescent state. Lymphangiogenesis, i.e., growth of new lymphatic vessels from the existing lymphatic vessels, will occur under some physiological and pathological conditions. Under physiological conditions, both corpus luteum development and wound healing will lead to lymphangiogenesis^{[1]}. Some pathological conditions, including tumor growth and metastasis, inflammation, and transplant rejection, can also cause the growth of lymphatic vessels^{[6]}. Although a few studies reported that bone marrow-derived cells, including macrophages, can be differentiated into lymphatic endothelial cells, lymphangiogenesis in adults primarily occurs by sprouting from existing lymphatic vessels to form new lymphatic vessels^{[7]}.

Tumor metastasis is the leading cause of cancer death. Tumor cells can metastasize through various pathways, including lymphatic vessels. Tumor cells metastasize to lymph nodes and distal organs through lymphatic vessels. Lymphatic metastasis of a tumor is often the first step in cancer cell spread and can be used as a primary diagnostic indicator of malignant tumor progression^{[8]}. Previous studies found that tumor tissues can activate lymphatic endothelial cells to induce the formation of new lymphatic vessels, i.e., tumor lymphangiogenesis. In animal models of tumor metastasis, tumor lymphangiogenesis can promote lymph node metastasis^{[9]}. More and more clinical data have also shown that in a variety of tumor types, tumor lymphangiogenesis is positively correlated with further tumor metastasis^{[10]}. Regarding how lymphangiogenesis is regulated, a series of growth factors have been discovered recently to induce lymphangiogenesis. Among those growth factors, Vascular Endothelial Growth Factor C (VEGF-C) and Vascular Endothelial Growth Factor D (VEGF-D) are the most important factors to promote lymphangiogenesis, which are glycoproteins and can activate Vascular Endothelial Growth Factor receptor 3 (VEGFR-3)^{[11, 12]}. VEGFR-3 is specifically expressed on adult lymphatic endothelial cells. The activation of VEGFR-3 can induce lymphatic endothelial cell proliferation in vitro and elicit lymphangiogenesis in vivo^{[13, 14]}. Conversely, in some human patients with hereditary lymphatic edema, tyrosine kinase domain cannot be activated due to missense mutation(s) in VEGFR-3, thereby affecting the signaling pathways^{[15]}. Similarly, artificial expression of a soluble VEGFR-3 fragment can antagonize VEGF-C and VEGF-D, thereby inhibiting lymphangiogenesis and causing lymphedema in transgenic mice^{[13]}. Full-length VEGF-C and VEGF-D can specifically act on lymphangiogenesis^{[16, 17]}, while mature-form fragments can induce the growth of both blood vessels and lymphatic vessels^{[18, 19]}.

Recently, a number of other lymphangiogenesis-related growth factors have been reported, for example, (1) Vascular Endothelial Growth Factor A (VEGF-A), which plays a role in lymphangiogenesis through the Vascular Endothelial Growth Factor Receptor 2 (VEGFR-2) pathway, as reported in Cueni et al.^{[20]}; (2) angiopoietin, whose tyrosine kinase receptor Tie-2 is specifically expressed on endothelial cells. Overexpression of angiopoietin-1 can induce lymphangiogenesis in a mouse cornea model^{[21]}. Simultaneous treatment of soluble VEGFR-3 fragments in mice can inhibit the functions of angiopoietin-1, which indicates that angiopoietin-1 plays a role through the VEGFR-3 pathway indirectly^{[21]}. In addition, angiopoietin-2 is a necessary factor for the development of lymphatic vessels. Angiopoietin-2 deficient mice lack normal lymphatic vessel tissues^{[22]}; (3) Hepatocyte Growth Factor (HGF), a recently discovered effective pro-lymphangiogenesis factor. Overexpression or intradermal administration of HGF in transgenic mice can promote lymphatic vessel hyperplasia, and anti-VEGFR-3 antibody cannot inhibit the activity of HGF, suggesting that HGF can promote lymphangiogenesis directly^{[23]}; (4) Basic Fibroblast Growth Factor (bFGF), which can also promote the growth of lymphatic vessels in a mouse cornea model, possibly through promoting the secretion of VEGF-C from vascular endothelial cells^{[24]}; (5) Platelet-Derived Growth Factor-BB (PDGF-BB), which, as reported in Cao et al., can promote the mobility of lymphatic endothelial cells in vitro and induce lymphangiogenesis in vivo in a mouse cornea model, and exerts its functions via Platelet-Derived Factor Receptor (PDGFR).

PDGF-BB-induced lymphangiogenesis can also be inhibited by a VEGFR-3 antagonist, suggesting that PDGF-BB can directly and indirectly act on lymphatic vessels^{[25]}; and (6) Insulin-like Growth Factor-1/2 (IGF-1/2), which can induce lymphangiogenesis in vivo, as reported in Bjorndahl et al.^{[26]}.

Human chemokine family currently comprises 40 chemokines and 18 chemokine receptors. Chemokines are about 8-15-kDa small molecule cytokines with chemotactic effects. According to the locations of conserved cysteines at N-terminal of amino acid sequences, chemokines are classified into four subgroups: CXC, CC, CX3C and C^{[27]}. Chemokines can play a role in chemotaxis by activating cell surface chemokine receptors to induce the directional cell migration towards a concentration gradient of chemokine. Chemokine receptors are usually seven-transmembrane G protein-coupled receptors on the surface of cell membrane. Chemokine receptors were initially found on the surface of immune cells, and they mediate the entry of immune cells into inflammation sites. Later, it was found that chemokine receptors were expressed on the surface of many hematogenous cells and non-hematogenous cells. Chemokine receptors expressed in different tissue microenvironments interact with their corresponding chemokines, and are responsible for assisting in coordinating transportation and organization of cells to a variety of tissues by means of chemotaxis^{[28, 29]}. In tumor tissues, chemokines can regulate tumor progression through influencing angiogenesis, interactions between tumor cells and inflammatory cells, or directly affecting tumor transformation, growth, invasion and metastasis.

Chemokine CXCL12, also known as Stromal-Derived Factor-1α (SDF-1α), can bind to chemokine receptor CXCR4^{[30]}. Chemokine CXCL12 is a highly conserved chemokine with 99% homology between human and mouse, allowing CXCL12 to act across species bathers. The chemokine CXCL12-chemokine receptor CXCR4 pathway can play a role in different species, such as zebrafish and mouse, in the course of evolution. Chemokine receptor CXCR4 is a rhodopsin-like G-protein-coupled receptor containing 352 amino acids^{[31]}. The initial study found that chemokine receptor CXCR4 plays a role in HIV infection and is a co-receptor of a certain HIV entering into CD4-positive T-cells^{[32]}, which led to extensive researches.

Chemokine CXCL12-chemokine receptor CXCR4 also play a significant role in the process of tumorigenesis. They mediate tumor cell metastasis to specific tissue organs^{[33]}. Chemokine CXCL12-chemokine receptor CXCR4 can promote tumor angiogenesis, assisting in tumor cell metastasis to specific organs, which has been reported in various tumor types, such as breast cancer, lung cancer, ovarian cancer, renal cancer, prostate cancer and glioma^{[34-39]}. Tumor cells expressing chemokine receptor CXCR4 tend to metastasize to the tissues with high chemokine CXCL12 expression, such as lung, liver, lymph nodes, bone marrow and other tissues. In breast cancer, tumor-associated fibroblasts can secrete a large amount of chemokine CXCL12 which can both directly promote the growth of breast cancer cells and promote angiogenesis to stimulate tumor growth. In addition, hypoxic environment is an important regulatory mechanism to change the behavior of tumor metastasis. As the oxygen concentration in tumor tissues is reduced, hypoxic environment can up-regulate the expression of chemokine receptor CXCR4 in tumor cells through Hypoxia-Inducible Factor 1α (HIF-1α)^{[40]}. Under normal physiological conditions, tumor suppressor protein Von Hippel-Lindau can down-regulate the expression of chemokine receptor CXCR4 through degradation of HIF-1α^{[41]}. Meanwhile, hypoxic environment can also increase the secretion of chemokine CXCL12 from tumor tissues, contributing to tumor cell survival. Chemokine CXCL12 is also involved in tumor cell invasion. Through up-regulating matrix metalloproteinase 13 (MMP13), chemokine CXCL12 promotes the invasion of human basal cell cancer cells^{[42]}. In view of the important roles of chemokine CXCL12 and chemokine receptor CXCR4 in tumors, they are very likely to be important targets for anti-cancer therapy. Chemokines and chemokine receptors play crucial roles in the processes of tumorigenesis, growth and metastasis, and thus the chemokine family can be considered as a potential target for the treatment of tumors. Some chemokines can promote tumor growth and metastasis, and some chemokines can inhibit tumor progression. The tumor growth, invasion and metastasis processes can be interfered with through the regulation of specific chemokines or chemokine receptors.

In summary, in tumor microenvironments, tumor tissues can activate lymphatic endothelial cells and lymphatic vessels to induce the formation of new lymphatic vessels from the existing ones, which is called tumor lymphangiogenesis. Tumor lymphangiogenesis is closely related to lymphatic metastasis. The newly formed lymphatic vessels provide a convenient metastasis pathway for tumor cells, therefore the tumor cells can metastasize through the lymphatic vessels to lymph nodes and distal organs. Animal experiments and clinical data have confirmed that in many tumor types, tumor lymphangiogenesis can serve as an indicator of lymph node metastasis. However, it has not yet been fully and clearly investigated how lymphangiogenesis is induced in tumor tissues and what the regulation mechanisms are. At present, it has been found that a series of growth factors secreted by tumor tissues, including Vascular Endothelial Growth Factor C (VEGF-C), the most important pro-lymphangiogenesis factor, can activate lymphatic endothelial cells and promote lymphangiogenesis. These growth factors activate lymphatic endothelial cells and promote their proliferation and migration, but it is not yet clear how these activated lymphatic endothelial cells are recruited to tumor tissues. The chemokine family comprises a variety of chemokines and chemokine receptors. Chemokines play a role in chemotaxis by activating the chemokine receptors expressed on specific cell surface to promote the directional cell migration towards a concentration gradient of chemokine, thereby the cells are recruited to specific tissues. Tumor tissues are also rich in the family of chemokines, some of which can promote tumor growth and metastasis. It is not yet clear whether the chemokines highly expressed in tumor tissues, especially under hypoxic conditions, can recruit the lymphatic endothelial cells activated by growth factors and thus participate in the regulation of tumor lymphangiogenesis.

### SUMMARY OF THE INVENTION

In the present invention, the inventors have completed the following studies.

The chemokine receptors expressed on the surface of lymphatic endothelial cells in the chemokine family were screened, and it was demonstrated that the lymphatic endothelia cells activated by vascular endothelial growth factor VEGF-C specifically up-regulated the expression of chemokine receptor CXCR4.

It was demonstrated that the ligand of CXCR4, chemokine CXCL12, was a new pro-lymphangiogenesis factor, which could directly act on lymphatic endothelial cells through chemokine receptor CXCR4 to recruit lymphatic endothelial cells in vitro and promote lymphangiogenesis in vivo.

It was demonstrated that chemokine CXCL12 directly functioned to promote lymphangiogenesis, independent of the vascular endothelial growth factor VEGF-C signaling pathway.

It was found that the multi-target combination treatment which inhibits both chemokine CXCL12 and growth factor VEGF-C pathways could more effectively inhibit tumor lymphangiogenesis and lymphatic metastasis.

These studies demonstrated that the chemokine family directly participated in the regulation of tumor lymphangiogenesis, proved that chemokine CXCL12 was a new pro-lymphangiogenesis factor, and found that the multiple-target combination treatment which blocks both chemokine pathway and growth factor pathway could more effectively inhibit lymphatic metastasis, which can become a new strategy for clinical inhibition of tumor lymphatic metastasis.

Based on these studies, the present invention provides a method for inhibiting lymphangiogenesis in a subject, comprising administering a therapeutically effective amount of a CXCR4 inhibitor and/or a CXCL12 inhibitor to the subject. The subject may suffer from tumor, inflammation and/or graft rejection reaction or the like. The CXCR4 inhibitor and CXCL12 inhibitor can be administrated individually or simultaneously.

The present invention further provides a method for inhibiting tumor lymphatic metastasis in a cancer patient, comprising administering a therapeutically effective amount of a CXCR4 inhibitor and/or a CXCL12 inhibitor to the subject.

The present invention further provides a method for inhibiting tumor lymphatic metastasis in a cancer patient, comprising administering to the subject (a) a therapeutically effective amount of a CXCR4 inhibitor and/or a CXCL12 inhibitor, in combination with (b) a therapeutically effective amount of a VEGF-C inhibitor and/or a VEGF-D inhibitor and/or a VEGFR-3 inhibitor.

In this method, (a) a CXCR4 inhibitor and/or a CXCL12 inhibitor are used to block CXCL12 pathway, and (b) a VEGF-C inhibitor and/or a VEGF-D inhibitor and/or a VEGFR-3 inhibitor are used to block VEGFR-3 pathway. The combined administration of the two types of substances can more effectively control tumor lymphatic metastasis.

It is worth noting that, in this method, the VEGF-C inhibitor, VEGF-D inhibitor and VEGFR-3 inhibitor as mentioned in (b) can be administrated individually or in a combination of the two or three.

In an embodiment of the present invention, intraperitoneal injection of chemokine CXCL12-neutralizing antibody and growth factor VEGF-C-neutralizing antibody into mice could effectively inhibit tumor lymphangiogenesis and tumor lymphatic metastasis.

In another aspect, the present invention provides use of a CXCR4 inhibitor and/or a CXCL12 inhibitor in the manufacture of a preparation for inhibiting lymphangiogenesis in a subject.

The present invention further provides use of a CXCR4 inhibitor and/or a CXCL12 inhibitor in the preparation of a medicament for inhibiting tumor lymphatic metastasis in a cancer patient.

The present invention further provides use of (a) a CXCR4 inhibitor and/or a CXCL12 inhibitor, and (b) a VEGF-C inhibitor and/or a VEGF-D inhibitor and/or a VEGFR-3 inhibitor in the preparation of a medicament for inhibiting tumor lymphatic metastasis in a cancer patient.

The present invention further provides a pharmaceutical composition for inhibiting tumor lymphatic metastasis in a cancer patient, comprising: (a) a CXCR4 inhibitor and/or a CXCL12 inhibitor, and (b) a VEGF-C inhibitor and/or a VEGF-D inhibitor and/or a VEGFR-3 inhibitor, as active ingredients; and optionally a pharmaceutically acceptable carrier.

The present invention further provides a kit for inhibiting tumor lymphatic metastasis in a cancer patient, comprising: (a) a CXCR4 inhibitor and/or a CXCL12 inhibitor, and (b) a VEGF-C inhibitor and/or a VEGF-D inhibitor and/or a VEGFR-3 inhibitor. The kit can further comprise instructions and an auxiliary means assisting in administering a medicament to a patient, such as syringe.

The tumors as described above include, but not limited to: brain astrocytoma, esophageal squamous cell carcinoma, gastric adenocarcinoma, hepatocellular carcinoma, colonic adenocarcinoma, rectal adenocarcinoma, lung squamous cell carcinoma, bladder urothelial carcinoma, cardiac myxoma, renal clear cell carcinoma, papillary thyroid carcinoma, pancreatic carcinoma, cervical squamous cell carcinoma, cutaneous squamous cell carcinoma, non-specific invasive ductal carcinoma of breast, ovarian clear cell carcinoma, prostate carcinoma and testicular seminoma.

### BRIEF DESCRIPTION OF THE DRAWINGS

FIG. 1.1 shows the results of the chemokine receptor expression in mouse lymphatic endothelial cells as detected by reverse transcription PCR. After total RNA was extracted from mouse lymphatic endothelial cells, the mRNA levels of chemokine family receptors were detected by reverse transcription PCR. As shown in the electropherograms of the PCR products, the chemokine receptor family includes CCR1-10, CXCR1-7 and CX3CR1, with GAPDH as a positive control. M=DNA marker; C=negative control without reverse transcriptase; T=target genes; bp=DNA molecular weight unit. The results showed that normally cultured mouse lymphatic endothelial cells expressed multiple chemokine receptors, among which CCR5, CCR9, CXCR4, CXCR6, and CXCR7 are highly expressed.

FIG. 2.1 shows the results of the chemokine receptor expression in VEGF-C-activated lymphatic endothelial cells as detected by qRT-PCR. qRT-PCR was conducted to detect the mRNA levels of chemokine receptors, including CCR4-6, CCR8-10, CXCR3-4, CXCR6-7, and CX3CR1, expressed in VEGF-C-stimulated mouse lymphatic endothelial cells, as compared with unstimulated cells. The results showed that VEGF-C-activated mouse lymphatic endothelial cells specifically up-regulated the expression of chemokine receptor CXCR4.

FIG. 2.2 shows the results of the CXCR4 expression up-regulated by VEGF-C as detected by flow cytometry. The expression levels of CXCR4 on the cell membrane surface of serum-starved cells and VEGF-C-stimulated cells were analyzed by flow cytometry. The expression of CXCR4 on the surface of lymphatic endothelial cells was up-regulated by VEGF-C stimulation.

FIG. 2.3 shows the results of the CXCR4 expression up-regulated by VEGF-C as detected by immunoblotting. After mouse lymphatic endothelial cells were treated with VEGF-C for the indicated time periods, the protein expressions of chemokine receptor CXCR4, hypoxia-inducible factor HIF-1α and the control, Lamin B, in the cells were detected by immunoblotting. The results showed that VEGF-C could up-regulate the expression of CXCR4 and HIF-1α.

FIG. 2.4 shows the effect of HIF-1α siRNA on CXCR4. After the expression of HIF-1α in mouse lymphatic endothelial cells was knocked down by siRNA, and the cells transfected with negative control siRNA (N.C.) or HIF-1α siRNA were stimulated by VEGF-C, the expressions of CXCR4, HIF-1α and the control, actin, in the cells were detected by immunoblotting. The results showed that the up-regulation of CXCR4 by VEGF-C was mediated by HIF-1α.

FIG. 3.1 shows the distribution of chemokine receptor CXCR4 in vivo. The expression of chemokine receptor CXCR4 (green) on the lymphatic vessels (Podoplanin, red) in colon and lymph node tissues from normal mice, and in tumor tissues and tumor-associated lymph node tissues from melanoma-bearing mice was observed under a laser confocal microscope. DAPI was used to stain nucleus (blue). Scale bar=20 μm. The results showed that the chemokine receptor is highly expressed on newly formed tumor-associated lymphatic vessels.

FIG. 3.2 shows the distribution of chemokine receptor CXCR4 in vivo. The expression of chemokine receptor CXCR4 on newly formed tumor lymphatic vessels in the colon tumor tissues, rectal tumor tissues and skin squamous cell carcinoma tissues from a normal person was observed under a laser confocal microscope. Lymphatic vessel (Podoplanin, red), chemokine receptor CXCR4 (green), and DAPI-stained nucleus (blue) are shown. Scale bar=200 μm. The results showed that the chemokine receptor is highly expressed on newly formed tumor-associated lymphatic vessels.

FIG. 4.1 shows the ability of chemokine CXCL12 to promote the migration of lymphatic endothelial cells. In the cell chemotaxis assay as shown in the figure, chemokine CXCL12 induces the migration of mouse lymphatic endothelial cells in a concentration-dependent manner. VEGF-C (100 ng/mL) was used as a positive control; ***, p<0.001.

FIG. 4.2 shows the ability of chemokine CXCL12 to promote the tubule formation of lymphatic endothelial cells. In the tubule formation assay as shown in the figure, chemokine CXCL12 induces the migration of mouse lymphatic endothelial cells in a concentration-dependent manner. VEGF-C (100 ng/mL) was used as a positive control; and ***, p<0.001.

FIG. 4.3 shows that chemokine CXCL12 promotes lymphangiogenesis. In the in vivo Matrigel plug assay as shown in the figure, the Matrigel mixed with different concentrations of chemokine CXCL12 was inoculated subcutaneously into mice, and then the Matrigel was removed for analysis of newly formed lymphatic vessels therein. Podoplanin represents lymphatic vessels (red), and DAPI represents stained nucleus (blue). CXCL12 can induce lymphangiogenesis in the mice in a concentration-dependent manner. VEGF-C was used as a positive control. The top panel of FIG. 4.3 shows the results of laser confocal microscopy, scale bar=200 nm; and the bottom panel of FIG. 4.3 shows the statistical results, ***, p<0.001.

FIG. 5.1 shows the effect of an antibody against chemokine receptor CXCR4 on signaling pathways in lymphatic endothelial cells. The effects of the anti-CXCR4 antibody on CXCL12-activated protein kinase B (Akt) and extracellular signal-regulated kinase (Erk) are shown in the figure. In the presence of the anti-CXCR4 antibody (5 μg/mL) or an isotype immunoglobulin as a control (IgG, 5 μg/mL), mouse lymphatic endothelial cells were treated with CXCL12 (100 ng/mL), and then the protein levels of Akt and Erk as well as their phosphoralation levels (p-Akt, p-Erk) were detected by immunoblotting.

FIG. 5.2 shows the effects of Erk and Akt antagonists on the recruitment of lymphatic endothelial cells by CXCL12. In the in vitro cell chemotaxis assay, mouse lymphatic endothelial cells were treated with the antagonists of Akt pathway (LY294002) and Erk pathway (U0126). The effects of these antagonists on the function of CXCL12 (100 ng/mL) in recruiting lymphatic endothelial cells were evaluated. The top panel of FIG. 5.2 shows the migrated mouse lymphatic endothelial cells (purple), scale bar=100 μm; and the bottom panel of FIG. 5.2 shows the statistical results of the migrated cells, ***, p<0.001.

FIG. 6.1 shows the results of the chemokine CXCL12 expression as detected on a human tumor tissue microarray. As shown in the figure, the human tumor tissue microarray comprises a variety of tumor types, a total of 54 specimens. The expression level of chemokine CXCL12 and the density of lymphatic vessels (LV) were detected by tissue immunofluorescence. The specimens were subsequently classified into 4 groups according to the signal strength. The number of specimens in each group was counted. The left panel of FIG. 6.1 is a representative graph showing the results of laser confocal microscopy, in which DAPI represents stained nucleus (blue); CXCL12 is stained in green; Podoplanin represents lymphatic vessels (red); co-localization represents the superposition of different fluorescences; scale bar=50 μm. The right panel of FIG. 6.1 shows the statistical results, in which CXCL12 (high) represents high expression of chemokine CXCL12; CXCL12 (low) represents low expression of chemokine CXCL12; LV (high) represents a high density of lymphatic vessels; and LV (low) represents a low density of lymphatic vessels.

FIG. 7.1 shows the effect of inhibiting CXCR4 on chemokine CXCL12. In the cell chemotaxis assay as shown in the figure, the cells were treated with anti-CXCR4 antibody and a CXCR4 antagonist (AMD3100). The activity of chemokine CXCL12 was inhibited, while the activity of VEGF-C was not affected. Cytokine is a control containing only chemokine CXCL12 or VEGF-C. IgG is an isotype immunoglobulin control. The top panel of FIG. 7.1 shows the cell migration results, scale bar=100 μm; and the bottom panel of FIG. 7.1 shows the statistical results, ***, p<0.001.

FIG. 7.2 shows the effect of inhibiting VEGFR-3 on chemokine CXCL12. In the cell chemotaxis assay as shown in the figure, the cells were treated with anti-VEGFR-3 antibody (VEGFR-3 Ab). The cell migration-promoting activity of chemokine CXCL12 was not affected, while the activity of VEGF-C was inhibited. IgG is an isotype immunoglobulin control; ***, p<0.001.

FIG. 7.3 shows the verification of the relationship between CXCL12 and VEGF-C by an in vivo Matrigel plug assay. The Matrigel mixed with corresponding agents as indicated in the figure was inoculated subcutaneously into mice. The formation of new lymphatic vessels in the Matrigel was detected by tissue immunofluorescence. AMD3100 is a CXCR4 antagonist; cytokine is a control containing only CXCL12 or VEGF-C; IgG is an isotype immunoglobulin control; DAPI represents stained nucleus (blue), and Podoplanin represents lymphatic vessels (red). The top panel of FIG. 7.3 shows the results of laser confocal microscopy, scale bar=50 μm; and the bottom panel of FIG. 7.3 shows the statistical results, ***, p<0.01.

FIG. 8.1 shows the additive effect of CXCL12 and VEGF-C as detected by a cell chemotaxis assay. In the cell chemotaxis assay as shown in the figure, mouse lymphatic endothelial cells were treated with chemokine CXCL12 and VEGF-C individually or simultaneously, and then the migration ability of the cells was detected. PBS was used as a control. The top panel of FIG. 8.1 shows the results of cell migration, scale bar=100 μm, and the bottom panel of FIG. 8.1 shows the statistical results, ***, p<0.001. Combination of chemokine CXCL12 and growth factor VEGF-C could promote lymphangiogenesis more effectively than each alone in vivo.

FIG. 8.2 shows the additive effect of CXCL12 and VEGF-C as detected by a cell chemotaxis assay. In the Matrigel plug assay as shown in the figure, combination of chemokine CXCL12 and growth factor VEGF-C could promote lymphangiogenesis more effectively than each alone in vivo.

FIG. 9.1 shows the density of lymphatic vessels in a human breast cancer nude mouse model. In the constructed enhanced Green Fluorescent Protein (eGFP)-labeled human breast cancer MDA-MB-231 cell nude mouse model, as shown in the figure, the mice were treated with anti-CXCL12 antibody and anti-VEGF-C antibody individually or in combination. The formation of new lymphatic vessels was detected by tissue immunofluorescence. IgG is an isotype immunoglobulin control; ***, p<0.001. The results showed that the combined blockage of chemokine CXCL12 and growth factor VEGF-C could inhibit lymphangiogenesis in tumor tissues more efficiently than blockage of either of the agents.

FIG. 10.1 shows the lymph nodes in human breast tumor-bearing nude mice. The figure shows the lymph node tissues from human breast tumor MDA-MB-231-bearing nude mice, six nude mice in each group, which were treated with anti-CXCL12 antibody and anti-VEGF-C antibody individually or in combination. IgG was used as an isotype immunoglobulin control. Peritumoral inguinal lymph nodes were isolated. Scale bar=2 cm. It can be seen that the swelling of mouse lymph nodes in agent treatment groups was significantly better than that in the control group.

FIG. 10.2 shows the lymph node metastasis of human breast cancer cells. Human breast cancer cell line (MDA-MB-231) was a cell line labeled with enhanced Green Fluorescent Protein (eGFP) (MDA-MB-231/eGFP), which can be directly observed in lymph node tissues under a laser confocal microscope. The left panel of FIG. 10.2 is an illustrative picture showing the results of the laser confocal microscopy, in which DAPI represents stained nucleus, 231/eGFP represents human breast cancer cells labeled with green fluorescent protein, i.e., MDA-MB-231/eGFP, scale bar=50 μm; in partial enlarged images, scale bar=20 μm. The right panel of FIG. 10.2 shows the statistical results, ***, p<0.001. The results showed that the combined blockage of CXCL12 and VEGF-C can inhibit tumor lymph node metastasis more efficiently.

### DETAILED DESCRIPTION OF THE INVENTION

The term “subject”, as used herein, refers to any mammal, e.g., mouse, rat, rabbit, dog, cattle, especially primate, such as human being. The “subject” may refer to a mammal suffering from a disease, for example, a mammal, especially a human, suffering from a cancer, or a healthy mammal without a disease. In certain preferred embodiments of the present invention, the “subject” is a human.

The term “optionally”, as used herein, means “may have or may not have”, “not essential”, or the like. For example, by “optionally a pharmaceutically acceptable carrier” is meant that the pharmaceutically acceptable carrier may or may not be included. It can be selected by a person skilled in the art according to the actual conditions.

The term “a therapeutically effective amount”, as used herein, refers to an amount of an active compound which is sufficient to elicit a biological or medical response in an animal or human as sought by a veterinarian or clinician. It should be appreciated that the dosage varies according to the compound to be administered, the administration route, the desired therapy and the condition of the subject. The typical daily dosage for a mammal to be treated ranges from 0.01 mg to 100 mg of an active ingredient per kg body weight, for example, 1 mg/kg or 2 mg/kg. If necessary, the daily dosage can be administered in divided doses. According to the principles well known in the art, the accurate amount of the active ingredient to be administered and the administration route depend on the body weight, age, gender of the subject and the specific condition being treated.

The active compounds of the present invention can be conveniently administered to a subject in a manner well known to a person skilled in the art, for example, oral administration, intravenous injection, intraperitoneal injection or intramuscular injection.

The present invention also provides a method for preparing the pharmaceutical composition of the invention, comprising mixing the active ingredient with an optional pharmaceutically acceptable carrier. The composition of the invention can be prepared using a conventional carrier well known in the art by a conventional method. Therefore, the composition for oral administration may comprise, for example, one or more of colorant, sweetener, flavoring agent and/or preservative.

The term “CXCR4”, as used herein, refers to chemokine receptor CXCR4, which is a rhodopsin-like G protein-coupled receptor containing 352 amino acids.

The term “CXCL12”, as used herein, refers to chemokine CXCL12, also known as Stromal-Derived Factor-1α (SDF-1α), which can bind to chemokine receptor CXCR4. Chemokine CXCL12 is a highly conserved chemokine with 99% homology between human and mouse, allowing chemokine CXCL12 to act across species barriers.

The term “VEGFR-3”, as used herein, refers to Vascular Endothelial Growth Factor Receptor-3, which is a tyrosine kinase receptor on the surface of cell membrane.

The term “VEGF-C”, as used herein, refers to Vascular Endothelial Growth Factor C, the major pro-lymphangiogenesis factor, which can activate Vascular Endothelial Growth Factor Receptor 3 (VEGFR-3).

The term “VEGF-D”, as used herein, refers to Vascular Endothelial Growth Factor D.

The term “CXCR4 inhibitor”, as used herein, refers to an agent which can specifically bind to CXCR4 and inhibit its biological functions, for example, an anti-CXCR4 antibody or an active fragment thereof, and a CXCR4 antagonist such as AMD3100.

The term “antibody”, as used herein, can be a monoclonal or polyclonal antibody.

The term “active fragment” of an antibody, as used herein, refers to a fragment which has binding specificity of the antibody. The active fragment of an antibody can be easily prepared by a person skilled in the art.

The term “CXCL12 inhibitor”, as used herein, refers to an agent which can specifically bind to CXCR12 and inhibit its biological functions, such as an anti-CXCL12 antibody or a CXCL12 antagonist or a soluble fragment of CXCR4 which can competitively bind to CXCL12.

The term “VEGFR-3 inhibitor”, as used herein, refers to an agent which can specifically bind to VEGFR-3 and inhibit its biological functions, for example, an anti-VEGFR-3 antibody or an antagonist which inhibits the activity of VEGFR-3 tyrosine kinase, such as SAR131675, MA751, BAY57-9352, Vandetanib, and the like.

The term “VEGF-C inhibitor”, as used herein, refers to an agent which can specifically bind to VEGF-C and inhibit its biological functions, such as an anti-VEGF-C antibody or a VEGF-C antagonist or a soluble fragment of VEGFR-3 or VEGFR-2 which can competitively bind to VEGF-C.

The term “VEGF-D inhibitor”, as used herein, refers to an agent which can specifically bind to VEGF-D and inhibit its biological functions, such as an anti-VEGF-D antibody or a VEGF-D antagonist or a soluble fragment of VEGFR-3 or VEGFR-2 which can competitively bind to VEGF-D.

In order to further illustrate the present invention in more details, examples of the present invention will be provided hereinafter with reference to the drawings. These examples are only provided for explanation and illustration purpose, and should not be construed as limiting the scope of the present invention.

### EXAMPLES

### Example 1

### Lymphatic Endothelial Cells Express a Variety of Chemokine Receptors

### Methods

1. Extraction of Total RNA from Cells and Detection of Chemokine Receptor Expression in Lymphatic Endothelial Cells by RT-PCR

The isolation and extraction of total RNA from cells was performed using TRIZOL reagent (purchased from Invitrogen) following the standard operations as described in the reagent instructions. 1 mL of TRIZOL was added to the primary lymphatic endothelial cells (about 1×10⁶) collected by centrifugation, repeatedly pipetted up and down for 30 times, and then set aside at room temperature for 5 minutes. After centrifugation at 10,000×g at 4° C. for 15 minutes, the supernatant was removed gently. 0.2 mL of chloroform was added to the supernatant, vortexed vigorously for about 15 seconds, and then set aside at room temperature for 3 minutes. After centrifugation at 10,000×g at 4° C. for 15 minutes, the sample was stratified into three layers, i.e., a yellow organic phase, an interphase layer, and a colorless aqueous phase. The desired RNA was contained in the aqueous phase, the volume of which is about 60% of that of the TRIzol reagent used. The aqueous phase was transferred into a fresh tube, to which 0.5 mL of isopropanol was added, mixed well, and set aside at room temperature for 10 minutes. After centrifugation at 10,000×g at 4° C. for 10 minutes, the supernatant was removed, and transparent gelatinous precipitate was found at the side and bottom of the tube. The precipitate was washed with 75% ethanol prepared in DEPC-treated water. After centrifugation at 7,500×g at 4° C. for 5 minutes, the supernatant was discarded. The precipitate was air-dried at room temperature and dissolved in 50 μl of DEPC water for use.

The synthesis of first strand cDNA was performed using a Fermentas kit (RevertAid™ First Strand cDNA Synthesis Kits) according to the standard instructions. 1 μg of RNA was used in a 20 μl reaction system. Oligo (dT)₁₅ provided in the kit was used as a primer. The program was run as follows: 42° C., 50 min; 95° C., 5 min; 4° C., 10 min. The reverse transcription product was used in subsequent PCR and fluorescence quantitative RT-PCR, and the rest of the product was stored in a refrigerator at −80° C.

PCR was conducted to detect the expression profiles of chemokine receptors in lymphatic endothelial cells. The PCR program was run as below: 40 cycles of denaturation at 95° C. for 30 s, annealing at 56° C. for 30 s, and extension at 72° C. for 40 s, in a 20 μL reaction system, followed by a final extension at 72° C. for 5 min GAPDH was used as an internal control. The PCR products were subjected to DNA electrophoresis and observation. The primers are listed as follows:

CCR1 forward primer (5′-3′):
(SEQ ID NO: 1)
CACCATCTTCCAGGAGCG
CCR1 reverse primer (5′-3′):
(SEQ ID NO: 2)
CAGTGAGCTTCCCGTTCAG
CCR2 forward primer (5′-3′):
(SEQ ID NO: 3)
GAGCCTGATCCTGCCTCTACTTG
CCR2 reverse primer (5′-3′):
(SEQ ID NO: 4)
CCTGCATGGCCTGGTCTAAGTGC
CCR3 forward primer (5′-3′):
(SEQ ID NO: 5)
GCTTTGAGACCACACCCTATG
CCR3 reverse primer (5′-3′):
(SEQ ID NO: 6)
TTCAGGCAATGCTGCCAGTCC
CCR4 forward primer (5′-3′):
(SEQ ID NO: 7)
CCAAAGATGAATGCCACAGAG
CCR4 reverse primer (5′-3′):
(SEQ ID NO: 8)
CGAACAGCAAATCCGAGATG
CCR5 forward primer (5′-3′):
(SEQ ID NO: 9)
GCTGAAGAGCGTGACTGAT
CCR5 reverse primer (5′-3′):
(SEQ ID NO: 10)
GAGGACTGCATGTATAATG
CCR6 forward primer (5′-3′):
(SEQ ID NO: 11)
GTGCCAATTGCCTACTCC
CCR6 reverse primer (5′-3′):
(SEQ ID NO: 12)
GGCTCACAGACATCACGATC
CCR7 forward primer (5′-3′):
(SEQ ID NO: 13)
TTCCAGCTGCCCTACAATGG
CCR7 reverse primer (5′-3′):
(SEQ ID NO: 14)
GAAGGTTGTGGTGGTCTCCG
CCR8 forward primer (5′-3′):
(SEQ ID NO: 15)
CAGGACCAGAGCCATCAAG
CCR8 reverse primer (5′-3′):
(SEQ ID NO: 16)
GATGTCATCCAGGGTGGAAG
CCR9 forward primer (5′-3′):
(SEQ ID NO: 17)
GCTGATCTGCTCTTTCTTG
CCR9 reverse primer (5′-3′):
(SEQ ID NO: 18)
GTGCTTGGATGACTTCTTGG
CCR10 forward primer (5′-3′):
(SEQ ID NO: 19)
GTACGATGAGGAGGCCTATTC
CCR10 reverse primer (5′-3′):
(SEQ ID NO: 20)
CGTGCGATGGCCACATAG
CXCR1 forward primer (5′-3′):
(SEQ ID NO: 21)
CGTCATGGATGTCTACGTGC
CXCR1 reverse primer (5′-3′):
(SEQ ID NO: 22)
GTAGCAGACCAGCATAGTG
CXCR2 forward primer (5′-3′):
(SEQ ID NO: 23)
AACAGTTATGCTGTGGTTGTA
CXCR2 reverse primer (5′-3′):
(SEQ ID NO: 24)
CAAACGGGATGTATTGTTACC
CXCR3 forward primer (5′-3′):
(SEQ ID NO: 25)
GAACGTCAAGTGCTAGATGCCTCG
CXCR3 reverse primer (5′-3′):
(SEQ ID NO: 26)
GTACACGCAGAGCAGTGCG
CXCR4 forward primer (5′-3′):
(SEQ ID NO: 27)
CTGTAGAGCGAGTGTTGC
CXCR4 reverse primer (5′-3′):
(SEQ ID NO: 28)
GTAGAGGTTGACAGTGTAG
CXCR5 forward primer (5′-3′):
(SEQ ID NO: 29)
CGAAGCGGAAACTAGAGCC
CXCR5 reverse primer (5′-3′):
(SEQ ID NO: 30)
CCAGCTTGGTCAGAAGC
CXCR6 forward primer (5′-3′):
(SEQ ID NO: 31)
CAGCTCTGTACGATGGGCAC
CXCR6 reverse primer (5′-3′):
(SEQ ID NO: 32)
CGGTTGAAGGCCTTGGTAGC
CXCR7 forward primer (5′-3′):
(SEQ ID NO: 33)
GACTATGCAGAGCCTGGC
CXCR7 reverse primer (5′-3′):
(SEQ ID NO: 34)
CTTATAGCTGGAGGTGCC
CX3CR1 forward primer (5′-3′):
(SEQ ID NO: 35)
GACGATTCTGCTGAGGCCTG
CX3CR1 reverse primer (5′-3′):
(SEQ ID NO: 36)
GCCCAGACTAATGGTGAC
GAPDH forward primer (5′-3′):
(SEQ ID NO: 37)
CAAGGTCATCCATGACAACTTTG
GAPDH reverse primer (5′-3′):
(SEQ ID NO: 38)
GTCCACCACCCTGTTGCTGTAG

### Results

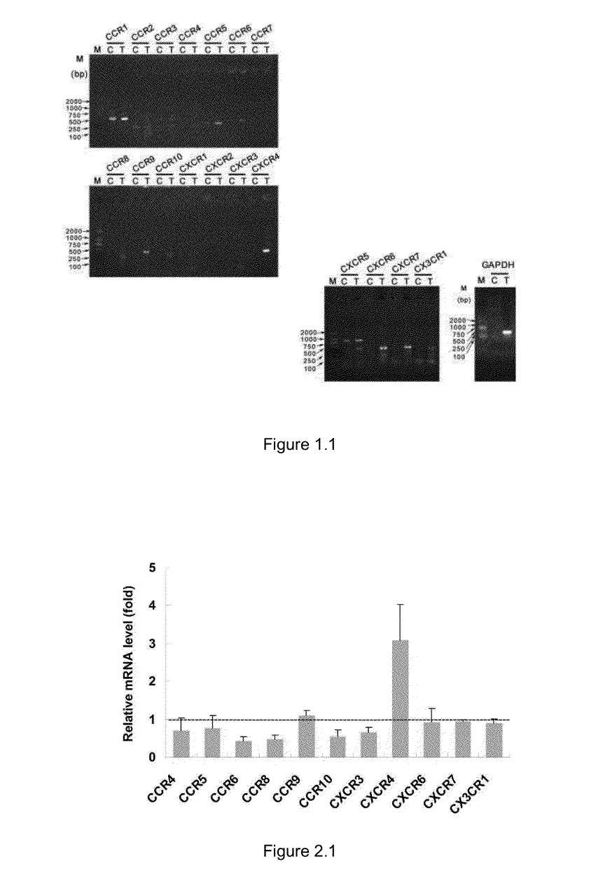

To investigate the roles of the chemokine receptor family in lymphangiogenesis, firstly, we need to determine which chemokine receptors are expressed on lymphatic endothelial cells. Chemokine receptors are G protein-coupled receptors expressed on the surface of specific cells, which can induce chemotactic response through binding to extracellular chemokine ligands, thereby promoting cells migration to specific locations. The chemokine receptors which have been found so far mainly include: CXCR1, CXCR2, CXCR3, CXCR4, CXCR5, CXCR6, CXCR7, CXCR8, CXCR9, CXCR10; CCR1, CCR2, CCR3, CCR4, CCR5, CCR6, CCR7; and CX3CR1. The levels of messenger RNAs (mRNAs) of those chemokine receptors in normally cultured mouse primary lymphatic endothelial cells were determined by semi-quantitative reverse transcription PCR. The PCR results showed that mouse lymphatic endothelial cells highly expressed chemokine receptors CCR5, CCR9, CXCR4, CXCR6 and CXCR7, and weakly expressed chemokine receptors CCR4, CCR6, CCR8, CCR10, CXCR3 and CX3CR1, but did not express other chemokine receptors (FIG. 1.1). The results demonstrated that lymphatic endothelial cells indeed expressed chemokine receptors, and the chemokine family might be involved in the regulation of the activities of lymphatic endothelial cells.

### Example 2

### Chemokine Receptor CXCR-4 is Highly and Specifically Expressed on VEGF-C-Activated Lymphatic Endothelial Cells

### Methods

### 1. Detection of the Expression of the Chemokine Receptors on Lymphatic Endothelial Cells by RT-PCR

Fluorescence Quantitative Real-Time PCR was performed using a kit from Stratagene (Brilliant II SYBR® Green QRT-PCR Master Mix). The fluorescence quantitative PCR instrument was MX3000P (purchased from Stratagene), the fluorescence dye was SYBR Green, the volume of the reaction system was 20 μL, and the cycle number of the reaction was 40. GAPDH was used as an internal control. The ΔCt value was obtained from the fluorogram provided by the instrument, and the relative Δ(ΔCt) value was calculated, thereby calculating the relative change in the level of the corresponding gene.

### 2. Verification of Vascular Endothelial Growth Factor C (VEGF-C)-Induced Expression of Chemokine Receptor CXCR4 on the Surface of Lymphatic Endothelial Cells by Flow Cytometry

Mouse primary lymphatic endothelial cells at passage 2 or 3 in good condition were selected and seeded into four 6 cm Petri dishes. Two groups of cells were cultured normally, while the other two groups of cells were treated by replacing the medium with a medium free of serum and growth factors when the cell density reached 80%, and starved overnight. One of the two groups was cultured for 24 hours after the medium was replaced with a serum-free culture medium containing 100 ng/mL of VEGF-C. The four groups of cells were detected for the expression level of chemokine receptor CXCR4 on the cell surface by flow cytometry. The group of cells cultured normally was used as negative control.

The cells were treated with 0.25% disodium EDTA (Ethylenediaminetetraacetic Acid Disodium Salt, EDTA), then washed with ice-cold PBS. The cells were suspended and centrifuged at a low speed (600 g, 3 minutes; the low-speed centrifugations in this experiment all referred to centrifugation at this speed for this time). The cells were resuspended in 1 mL of PBS containing 10% goat serum and then incubated for 15 minutes.

Each group of cells were centrifuged at a low speed and then resuspended in 1 mL of PBS containing 2% goat serum. The cells were incubated with a control antibody in the negative control group and with 1 μg of CXCR4 antibody (purchased from Abcam) in the other three groups, for 30 minutes. The cells were centrifuged at a low speed and then resuspended in 1 mL of PBS. This step was repeated once to remove the unbound antibody.

Each group of cells were resuspended in 1 mL of PBS containing 2% goat serum, and then 1 μg of fluorescein-labeled secondary antibody was added. The cells were centrifuged at a low speed and then resuspended in 1 mL of PBS. This step was repeated once to remove the unbound secondary antibody. Finally, the cells were resuspended in 500 μL of PBS. The expression of CXCR4 on cell surface was analyzed by flow cytometry (FACS Calibur Flow Cytometry System, Becton Dickinson).

### 3. Detection of VEGF-C-Induced Expression of Chemokine Receptor CXCR4 on the Surface of Lymphatic Endothelial Cells by Immunoblotting (IB)

Mouse primary lymphatic endothelial cells at passage 2 or 3 in good condition were selected, and starved overnight by replacing the culture medium with a fresh medium free of serum and growth factors. Then the medium was replaced with a medium containing 100 ng/mL of VEGF-C. The cells were incubated for 6, 12 and 24 hours, respectively, and then trypsinized and collected by centrifugation for detecting the expression level of chemokine receptor CXCR4 in the cells by immunoblotting.

The samples were subjected to SDS-PAGE (the concentration of separation gel was 15%). Protein bands were transferred to polyvinylidene difluoride (PVDF) membrane (purchased from Millipore) at 100 mA for 3 hours using an electroblotting device in ice bath. TBST buffer (20 mM Tris, pH7.4, 150 mM NaCl, 0.1% Tween-20) was formulated for preparing blocking solution, primary antibody solution and secondary antibody solution. The PVDF membrane was further incubated in blocking solution containing 5% skim milk for 1 hour at room temperature with gentle shaking. The membrane was then washed with TBST for 5 times, 5 minutes each.

According to the manufacturer's instructions, primary antibodies were diluted with TBST buffer containing 1% skim milk to provide primary antibody dilutions. The primary antibodies include anti-CXCR4 antibody (purchased from Abcam), anti-hypoxia-inducible transcription factor 1α (HIF-1α) antibody (purchased from Santa Cruz Biotechnology), anti-Lamin B antibody (purchased from Santa Cruz Biotechnology) and anti-Actin antibody (purchased from Santa Cruz Biotechnology). Each antibody was incubated with PVDF membrane overnight at 4° C. with gentle shaking. The membrane was then washed with TBST for 5 times (5 minutes each) at room temperature.

According to the manufacturer's instructions, horseradish peroxidase-conjugated secondary antibodies corresponding to specific species were diluted with TBST buffer containing 1% skim milk to provide secondary antibody dilutions. The PVDF membrane was incubated with an appropriate secondary antibody for 1 hour at room temperature with shaking, and then washed with TBST for 5 times (5 minutes each). The membrane was imaged by using the ECL detection kit (SuperSignal West Pico/Femto Chemiluminescent Substrate, Thermo Scientific). The X-ray film (purchased from Kodak) was exposed, developed and fixed in the dark. The results were scanned and saved.

4. RNA Interference of HIF-1α Verifies that HIF-1α is Involved in VEGF-C-Induced Upregulation of CXCR4 Expression

The siRNA for interfering with the chemical synthesis of HIF-1α was purchased from Santa Cruz Biotechnology, and the negative control siRNA was purchased from GenePharma, Shanghai. The transfection reagent for RNA interference was Lipofectamine 2000 (Invitrogen). The transfection was conducted according to the manufacturer's instructions for the transfection reagent. Mouse primary lymphatic endothelial cells at passage 2 or 3 in good condition were selected and seeded into 6-well plates. The cells were cultured for 24 hours until the cell density reached 40-50%. The culture medium was replaced with serum-free ECM (Sciencell) 30 minutes before transfection. Transfection solution was then prepared as follows. 100 nM siRNA was added to 100 μL ECM, mixed gently, and set aside at room temperature for 5 minutes. 8 μL of Lipofectamine 2000 was diluted in 100 μL ECM, mixed gently, and set aside at room temperature for 5 minutes. The siRNA dilution was slowly added into the transfection reagent dilution dropwise, mixed gently, and set aside at room temperature for 15 minutes. The as-prepared siRNA transfection solution was slowly added into 6-well plates dropwise while shaking the 6-well plates gently to result in even distribution. After 6-hour normal incubation in a cell incubator, an equal amount of serum-free medium was added and the cells were cultured under normal condition in the incubator overnight. The medium was replaced with normal ECM containing 10% FBS and the cells were cultured for additional 36-48 hours. HIF-1α knock-down efficiency was detected by immunoblotting.

The mouse primary lymphatic endothelial cells transfected with HIF-1α siRNA or negative control siRNA were starved overnight by replacing the medium with serum-free ECM. Then, the medium was replaced with serum-free ECM only containing 100 ng/mL VEGF-C and serum-free ECM without VEGF-C. The cells were incubated in an incubator for 6 hours and then collected for immunoblotting to detect CXCR4 and HIF-1α expression.

### Results

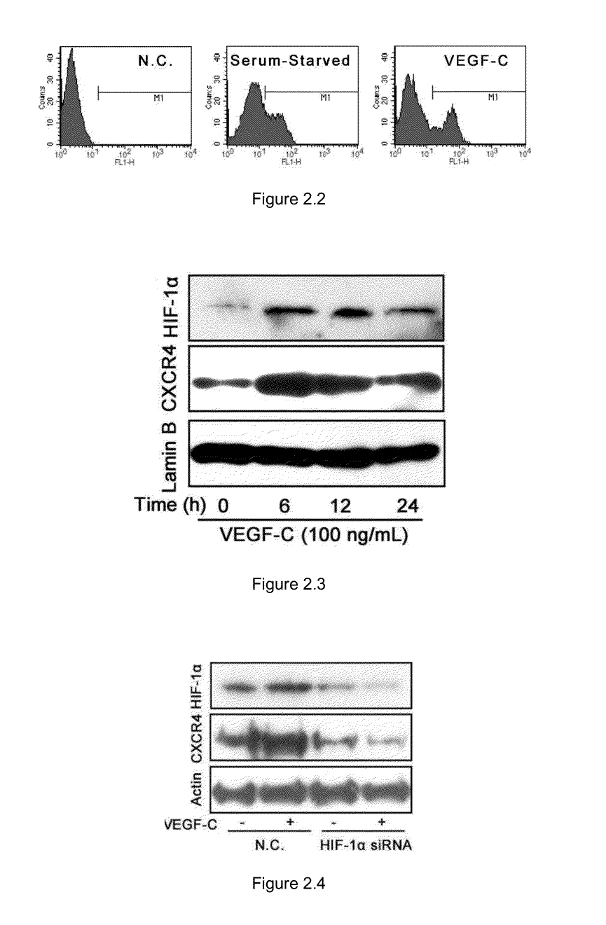

Since tumor tissues secrete a variety of growth factors to activate lymphatic endothelial cells, promoting their proliferation and migration, as well as lymphangiogenesis, whether the lymphatic endothelial cells activated by growth factors express abnormal chemokine receptors and what are the chemokine receptor expression profiles in the activated lymphatic endothelial cells need to be investigated. Vascular Endothelial Cell Growth Factor C (VEGF-C) is the most important pro-lymphangiogenesis factor among the factors which have been found in tumor tissues. In this experiment, mouse lymphatic endothelial cells were treated with VEGF-C, and the change in mRNA level of the chemokine receptor that can be expressed in lymphatic endothelial cells was detected by fluorescence quantitative real-time PCR (qRT-PCR). The results of fluorescence quantitative real-time PCR showed that, compared with untreated cells, when the mouse lymphatic endothelial cells were activated by VEGF-C, only the chemokine receptor CXCR4 mRNA levels were significantly upregulated by about three times, while other chemokine receptors showed no change (FIG. 2.1).

The results of fluorescence quantitative real-time PCR showed that VEGF-C could specifically upregulate the level of the chemokine receptor CXCR4 at mRNA level. The results were further confirmed at protein level. Mouse primary lymphatic endothelial cells were starved overnight and then treated with VEGF-C. The cell surface CXCR4 protein levels were detected by flow cytometry. The results showed that VEGF-C treatment could indeed upregulate the expression level of chemokine receptor CXCR4 in mouse lymphatic endothelial cells (FIG. 2.2). The immunoblotting assay also produced similar results, i.e., the treatment of mouse lymphatic endothelial cells with CXCR4 could upregulate the expression level of CXCR4 (FIG. 2.3). How does VEGF-C upregulate the expression level of CXCR4? VEGF-C, as an extracellular growth factor, may regulate the expression of chemokine receptor CXCR4 by regulating corresponding transcription factors. It has been reported that the gene of chemokine receptor CXCR4 is one of the target genes of Hypoxia-Inducible Transcription Factor 1α (HIF-1α)^{[41]}. Our immunoblotting results showed that VEGF-C could indeed upregulate the expression level of HIF-1α in mouse lymphatic endothelial cells (FIG. 2.3).

To further verify that HIF-1α is involved in the upregulation of chemokine receptor CXCR4 expression in lymphatic endothelial cells by VEGF-C, RNA interference method was used to knockdown the level of HIF-1α in mouse lymphatic endothelial cells. Immunoblotting results showed that VEGF-C could induce the expression of chemokine receptor CXCR4 in the mouse lymphatic endothelial cells transfected with negative control siRNA. When HIF-1α was knocked down by siRNA in the cells, VEGF-C stimulation could not induce the expression of chemokine receptor CXCR4 (FIG. 2.4). The results showed that HIF-1α was involved in the upregulation of chemokine receptor CXCR4 expression in mouse lymphatic endothelial cells by VEGF-C.

### Example 3

### CXCR4 is Highly and Specifically Expressed on the Surface of Newly Formed Lymphatic Vessels In Vivo

### Methods

### 1. Detection of the Distribution of Chemokine CXCR4 on Lymphatic Vessels In Vivo by Tissue Immunofluorescence

Eight healthy C57BL/6 mice (6-8 weeks old, female, purchased from Vital River Laboratories, Beijing) were divided into two groups, one of which includes three mice normally fed, and the other includes five tumor-bearing mice intracutaneously inoculated with 5×10⁶ B16/F10 mouse melanoma cells (American Type Culture Collection, ATCC). 14 days after inoculation, the tumor tissues and peritumoral axillary lymph node tissues were removed from the tumor-bearing mice, and the colon tissues and lymph node tissues were removed from the normal mice.

Fixing and embedding: The removed tissues were fixed with 4% formaldehyde solution overnight, and then rinsed with tap water overnight to completely wash off formaldehyde (the tissue blocks could be wrapped with gauze, placed in a beaker, and washed with water dripping from the tap overnight). The tissues washed overnight was dehydrated with alcohol at the following concentration gradient: 50% ethanol, 70% ethanol, 80% ethanol, 90% ethanol, and 95% ethanol, once for each step, 2 hours each time; dehydrated twice in 100% ethanol, 1 hour each time; dehydrated twice in xylene, 1 hour each time; and dehydrated twice in liquid paraffin (60° C.), 30 minutes each time. The dehydrated tissue blocks were embedded in paraffin and the embedded tissue blocks could be kept at room temperature for long-term storage. The paraffin-embedded tissue blocks were sliced into 8 nm thick sections by a microtome, flattened in water at 37° C., and then mounted on anti-shedding glass slides (Zhongshan Golden Bridge Company). The slides were baked in a dry environment at 55° C. for 2 hours or at 37° C. overnight.

Tissue rehydration and antigen retrieval: The baked sections were deparaffinized and rehydrated in the following order: twice in xylene, 3 minutes each time; twice in 100% ethanol, 2 minutes each time; in 95% ethanol, 90% ethanol, 80% ethanol, 70% ethanol, once for each step, 2 minutes each time. After being washed in PBS, the sections were thermally retrieved in sodium citrate. Then, the tissue sections were mounted on a slice rack and placed in a large beaker containing about 1 L of 10 mM sodium citrate retrieval solution (pH=6.0), such that the tissue sections were immersed in the retrieval solution at a level at least 2 cm lower than the liquid level. The beaker was heated in a microwave oven until just boiling (note: avoid boiling violently to prevent the tissues from detaching from the slide), and then maintained for 15 minutes after the microwave oven was adjusted to the “defrost” setting, i.e., at a water temperature between 90° C. and 95° C. The beaker was removed, slowly cooled down to room temperature (the cooling time was about 1 hour), and then washed with PBS.

Tissue immunofluorescence staining: The tissue sections subjected to antigen retrieval were blocked with a blocking solution in a humidified chamber at room temperature for 1 hour. The blocking solution was PBS containing 10% normal goat serum. Then the blocking solution was removed. Anti-Podoplanin primary antibody and anti-CXCR4 primary antibody (purchased from Abcam) were directly added according to the antibody instructions. The antibody diluting buffer was PBS containing 1% normal goat serum. The sections were incubated in a humidified chamber at room temperature for 1 hour. The primary antibodies were removed and the sections were washed with PBS for five times, 5 minutes each time. Fluorescein-labeled secondary antibodies were added according to the antibody instructions. The antibody diluting buffer was PBS containing 1% normal goat serum. The sections were incubated in a humidified chamber at room temperature for 1 hour. The secondary antibodies were removed and the sections were washed with PBS for three times, 5 minutes each time. DAPI was used to stain the nucleus. Then the sections were washed with PBS for 2 times, 5 minutes each time. The sections were mounted with Clearmount (Beijing Zhongshan Golden Bridge Corporation), a water soluble mounting agent, and then observed and imaged by a laser confocal microscope (Nikon A1) using the imaging software NIS-Elements AR 3.0.

The immunofluorescence staining method used for human tumor tissues was the same as above.

### Results

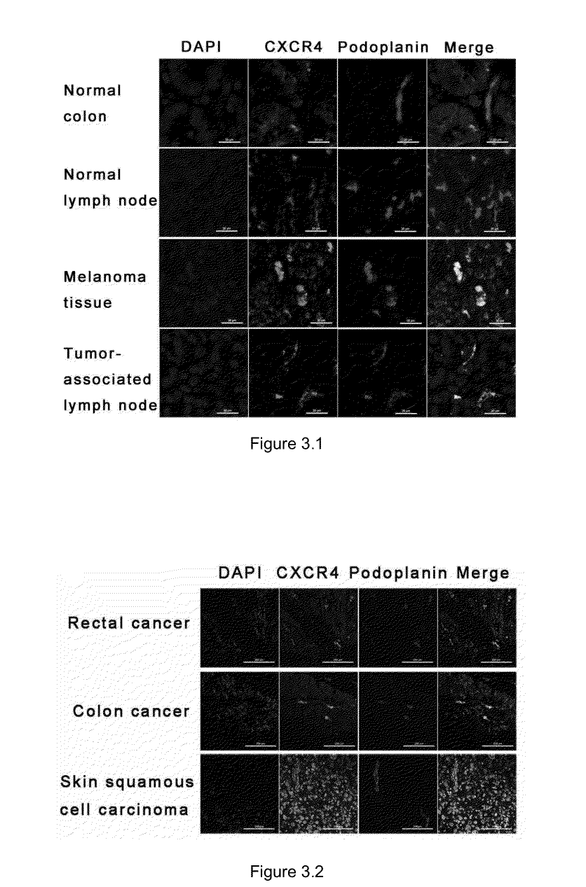

The results showed that when mouse lymphatic endothelial cells were activated by VEGF-C, the expression of chemokine receptor CXCR-4 was specifically up-regulated. We further detected the distribution of chemokine receptor CXCR4 on lymphatic vessels in vivo, especially the expression differences between normal mature lymphatic vessels and newly formed lymphatic vessels. The results of tissue immunofluorescence showed that no chemokine receptor CXCR4 was expressed on the mature lymphatic vessels in the normal mouse tissues such as colon tissues and lymph node tissues, whereas chemokine receptor CXCR4 was highly expressed on the tumor-associated lymphatic vessels in the mouse melanoma tumor tissues and the sentinel lymph node tissues of tumor-bearing mice (FIG. 3.1). A high expression of chemokine receptor CXCR4 on newly formed tumor-associated lymphatic vessels was also found when we examined human tumor tissues, including human colon cancer tissue, rectal cancer tissue and skin squamous cell carcinoma tissue (FIG. 3.2). It was possible that the expression of chemokine receptor CXCR4 was up-regulated in tumor-activated newly formed lymphatic vessels, which is consistent with our in vitro results, indicating that the expression of chemokine receptor CXCR4 on newly formed lymphatic vessels was up-regulated.

### Example 4

### Chemokine CXCL12 is a New Pro-Lymphangiogenesis Factor

### Methods

### 1. Cell Chemotaxis Assay

The migration ability of mouse lymphatic endothelial cells was assessed using 8-μm-pore Transwell bucket (filter membrane) (purchased from Costar). The transwell bucket was placed into a 24-well plate. Mouse primary lymphatic endothelial cells (mLECs) at passage 2 or 3 in good conditions were selected, and divided into five groups, four parallel samples for each group and approximately 2×10⁴ cells for each parallel sample. The cells were digested with trypsin, resuspended in 200 μL of fresh serum-free endothelial cell culture medium (ECM, purchased from Sciencell), and then seeded into the inner chambers of the Transwell bucket. To each outer chamber was added 800 μL of serum-free endothelial cell culture medium mixed with 1 ng/mL, 20 ng/mL, 100 ng/mL of chemokine CXCL12 (purchased from R&D Systems), 100 ng/mL of VEGF-C (purchased from R&D Systems) and PBS control, respectively. The culture plate was placed into an incubator and incubated under 5% CO₂ at 37° C. for 6 hours.

After taking out the culture plate, the Transwell bucket was fixed in 4% paraformaldehyde solution for 15 minutes. The Transwell bucket was rinsed twice in PBS, stained with 0.1% crystal violet solution for 30 minutes, and then washed with PBS to remove the non-specifically bound crystal violet. The cells inside Transwell membrane were wiped off gently with medical cotton swabs. Note that the cells on the edge of the membrane should also be wiped off to avoid affecting cell counting. The Transwell bucket was placed under a microscope (Olympus IX71 microscope), and the cells migrated from the outer membrane were observed by microscopy. 8 fields per group were photographed randomly to count the cells.

### 2. Tubule Formation Assay

A 24-well cell culture plate was pre-coated with a layer of growth factor-free Matrigel (purchased from Becton-Dickinson Biosciences, catalog No. 354230), approximately 150 μl per well, and set aside at 37° C. for 30 minutes until the Matrigel was coagulated. Mouse primary lymphatic endothelial cells at passage 2 or 3 in good conditions were selected, and seeded into the 24-well culture plate coated with the Matrigel, approximately 2×10⁴ cells per well. There were five groups, three parallel samples for each group. The medium was fresh serum-free ECM medium, containing 1 ng/mL, 20 ng/mL, 100 ng/mL of chemokine CXCL12 (purchased from R&D Systems), 100 ng/mL of VEGF-C (purchased from R&D Systems) and PBS control, respectively, for each group. The culture plate was placed into an incubator and incubated under 5% CO₂ at 37° C. for 6 hours. The endothelial cells would be spontaneously connected to form a tubule-like structure in the presence of extracellular matrix. The reticular structure formed by mouse lymphatic endothelial cells was observed under a Olympus microscope (Olympus IX71 microscope) and the length of the reticular structure, which indicates the ability of mouse lymphatic endothelial cells to form a tubule-like structure, was recorded by NIH Image J software^{[43]}.

### 3. In Vivo Matrigel Plug Assay

Matrigel plug assay was conducted as previously reported^{[44]}. BALB/C mice (5 weeks old, female, purchased from Vital River Laboratories, Beijing) were divided into five groups, five mice each group. Growth factor-free Matrigel (9-10 mg/mL, purchased from Becton-Dickinson Biosciences) containing 20 ng/mL, 100 ng/mL, 500 ng/mL of chemokine CXCL12 (purchased from R&D Systems), 500 ng/mL of VEGF-C (purchased from R&D Systems), and PBS control, respectively, was injected subcutaneously into the BALB/c mice along the abdominal midline. The Matrigel formed a solid plug in the mice, and the agent was slowly released from the Matrigel to stimulate new mouse lymphatic vessels to be formed and grown into the Matrigel. 8 days later, the Matrigel was removed carefully.

After being washed in PBS, the Matrigel were fixed in 30% sucrose solution at 4° C. overnight. Frozen sections in a thickness of about 10 nm were prepared and stored at −20° C. The frozen sections were blocked with a blocking solution in a humidified chamber at room temperature for 1 hour. The blocking solution was PBS containing 10% normal goat serum. After the blocking solution was removed, anti-Podoplanin primary antibody (purchased from Santa Cruz Biotechnology) was added directly according to the antibody instructions. The antibody diluting buffer was PBS containing 1% normal goat serum. The sections were incubated in a humidified chamber at room temperature for 1 hour. The primary antibody was removed and the sections were washed with PBS for 3 times, 5 minutes each time. Fluorescein-labeled secondary antibody was added according to the antibody instructions. The antibody diluting buffer was PBS containing 1% normal goat serum. The samples were incubated in a humidified chamber at room temperature for 1 hour. The secondary antibody was removed and the samples were washed with PBS for 3 times, 5 minutes each time. DAPI was used to stain the nucleus. Then the samples were washed twice with PBS, 5 minutes each time. The samples were mounted with Clearmount (Beijing Zhongshan Golden Bridge Corporation), and then observed and imaged by a laser confocal microscope (Nikon A1) using the imaging software NIS-Elements AR 3.0.

### Results

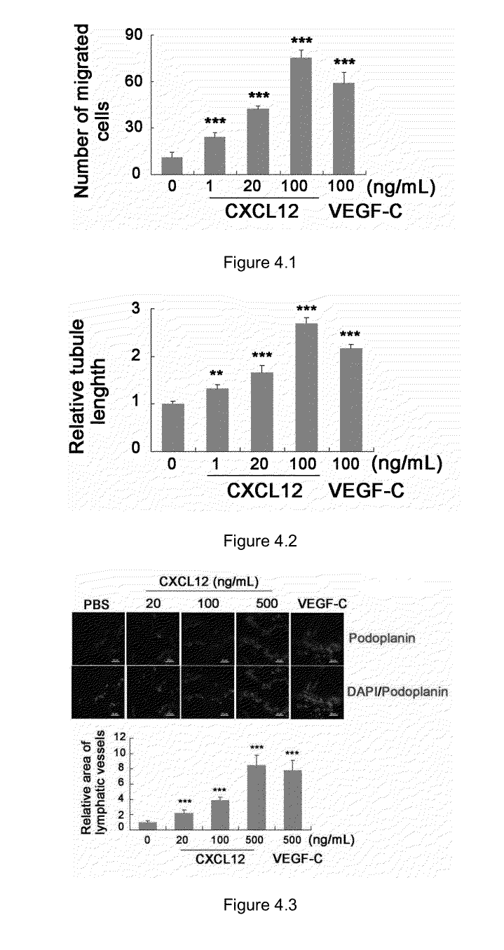

The above results showed that chemokine receptor CXCR4 was highly expressed on the surface of mouse lymphatic endothelial cells, and the expression of CXCR4 was up-regulated when the cells were activated by VEGF-C. Therefore, it was inferred that chemokine CXCL12, a ligand of CXCR4, could directly act on mouse lymph endothelial cells to promote their migration. Thus, we established an in vitro lymphatic endothelial cell chemotaxis model. The Transwell™ experiment results showed that different concentrations of chemokine CXCL12 significantly promoted the migration of mouse lymph endothelial cells (FIG. 4.1).

During lymphangiogenesis, newly formed lymphatic vessels proliferate by “sprouting” from the existing lymphatic vessels, and the migrated and the proliferated lymphatic endothelial cells establish connections between each other to form a lymphatic tubule. The formation of a tubule-like structure in vitro is an important feature of endothelial cells, as well as an important step for lymphangiogenesis. We studied the effect of chemokine CXCL12 on lymphangiogenesis in vitro from the point of view of the ability to form a tubule-like structure. The results showed that chemokine CXCL12 significantly promoted mouse lymphatic endothelial cells to form a regular tubule structure in a concentration-dependent manner in the petri dish coated with Matrigel (FIG. 4.2).

The in vitro experiments demonstrated that chemokine CXCL12 could directly act on mouse lymphatic endothelial cells and promote the migration and tubule formation abilities of lymphatic endothelial cells, both of which are important steps for lymphangiogenesis. Then, can chemokine CXCL12 promote lymphangiogenesis in vivo? We conducted a Matrigel plug assay. The Matrigel mixed with chemokine CXCL12 was injected subcutaneously into mice to induce lymphangiogenesis. After a period of time, the Matrigel plug was removed, and the newly formed lymphatic vessels in the Matrigel was examined. The immunofluorescence results showed that the Matrigel mixed with chemokine CXCL12 could recruit more mouse lymphatic endothelial cells to form a clear tubule structure in a concentration-dependent manner (FIG. 4.3). The statistical results also proved that chemokine CXCL12 could effectively induce lymphangiogenesis.

### Example 5

### CXCL12 Activates the Relevant Signaling Pathways in Lymphatic Endothelial Cells

### Methods

### 1. Effect of Antibody-Blockage of CXCR4 on the Chemokine CXCL12 Signaling Pathway

Mouse primary lymphatic endothelial cells at passage 2 or 3 in good conditions were selected and divided into four groups. The medium was replaced with serum-free ECM the night before treatment and the cells were starved overnight. One group of cells were cultured in serum-free ECM as a control, and the other 3 groups of cells were pretreated in serum-free ECM containing CXCR4-neutralizing antibody (5 μg/mL, purchased from Bioss, Beijing), isotype IgG control (5 μg/mL, prepared in the laboratory) or PBS control for 30 minutes. To the 3 treatment groups of cells was added 100 ng/mL CXCL12 (purchased from R & D Systems), and treated for 10 minutes. The cells were collected to detect the phosphorylation levels of protein kinase B (Akt) and extracellular signal-regulated kinase (Erk) in the cells by immunoblotting.

### 2. Effect of Inhibition of Signaling Pathway on the Functions of Chemokine CXCL12

The migration ability of mouse lymphatic endothelial cells was assessed using 8-μm-pore Transwell bucket (purchased from Costar). The bucket was placed into a 24-well plate. Mouse primary lymphatic endothelial cells (mLECs) at passage 2 or 3 in good conditions were selected, and divided into six groups, four parallel samples for each group, and approximately 2×10⁴ cells for each parallel sample. The cells were digested with trypsin and resuspended in 200 μL of fresh serum-free endothelial cell culture medium (ECM, purchased from Sciencell). The cells were pretreated for 30 minutes with dimethyl sulfoxide (DMSO) as a control, protein kinase B (Akt) antagonist LY294002 (10 μM, purchased from Sigma-Aldrich) and extracellular signal-regulated kinase (Erk) antagonist U0126 (10 μM, purchased from Sigma-Aldrich), respectively, and then seeded into the inner chambers of the Transwell bucket. To each outer chamber was added 800 μL of serum-free endothelial cell culture medium (ECM) mixed with dimethyl sulfoxide (DMSO) as a control, protein kinase B (Akt) antagonist LY294002 (10 nM) and extracellular signal-regulated kinase (Erk) antagonist U0126 (10 nM), respectively. Simultaneously, 100 ng/mL of chemokine CXCL12 (purchased from R&D Systems) was added to another group of cells. The culture plate was placed into an incubator and incubated under 5% CO₂ at 37° C. for 6 hours. The cells were stained and the number of the migrated cells was counted.

### Results

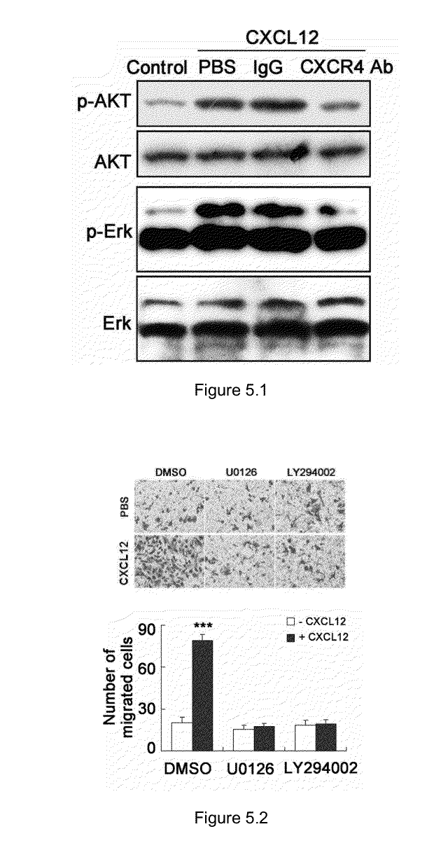

The in vitro experiments showed that chemokine CXCL12 could recruit lymphatic endothelial cells and promoted tubule formation ability of lymphatic endothelial cells. The in vivo experiments demonstrated that lymphangiogenesis could be promoted by chemokine CXCL12. We further tested the relevant cell signaling pathways in mouse lymphatic endothelial cells. Hu's et al. found that in myocardial cells, the phosphorylation of protein kinase B (Akt) and extracellular signal-regulated kinase (Erk) could be activated by chemokine CXCL12^{[45]}. In our mouse lymphatic endothelial cell model, consistent results were obtained by immunoblotting, i.e., chemokine CXCL12 could activate protein kinase B (Akt) and extracellular signal-regulated kinase (Erk) in mouse lymphatic endothelial cells, but did not affect their protein levels (FIG. 3.5). Then, is the activation of protein kinase B (Akt) and extracellular signal-regulated kinase (Erk) pathways in mouse lymphatic endothelial cells by chemokine CXCL12 mediated by chemokine receptor CXCR4? We used CXCR4-neutralizing antibody to block chemokine CXCR4, and then used chemokine CXCL12 to stimulate mouse lymphatic endothelial cells. The immunoblotting results showed that CXCR4-neutralizing antibody also inhibited the activity of chemokine CXCL12. The protein kinase B (Akt) and extracellular signal-regulated kinase (Erk) pathways could not be activated by chemokine CXCL12, while in the isotype IgG control group, the phosphorylation of protein kinase B (Akt) and extracellular signal-regulated kinase (Erk) could still be stimulated by chemokine CXCL12 (FIG. 5.1).

The above results showed that the signaling pathways of protein kinase B (Akt) and extracellular signal-regulated kinase (Erk) pathways in mouse lymphatic endothelial cells could be activated by chemokine CXCL12. Then, whether the promotion of lymphatic endothelial cell migration by chemokine CXCL12 is mediated by protein kinase B (Akt) and extracellular signal-regulated kinase (Erk)? In chemotaxis assays, we used protein kinase B (Akt) pathway antagonist LY294002 and extracellular signal-regulated kinase (Erk) inhibitor U0126 to treat cells, respectively. The two antagonists also inhibited the lymphatic endothelial cell migration induced by chemokine CXCL12 (FIG. 5.2). The results showed that protein kinase B (Akt) and extracellular signal-regulated kinase (Erk) pathways were involved in the promotion of lymphangiogenesis by chemokine CXCL12.

### Example 6

### The Expression Level of CXCL12 is Positively Correlated with Lymphangiogenesis in Human Tumor Tissues

### Methods

### 1. Detection of CXCL12 Expression Level and Lymphatic Vessel Density in Human Tumor Tissue Microarray by Immunofluorescence

A human multi-tumor tissue microarray, which was purchased from Xi'an Aomei, contains 54 clinical specimens, wherein the average age of the subjects is 55.6 years, ranging from 15 to 81 years; and the ratio of male to female is 31:23. The tumor types includes brain astrocytoma, esophageal squamous cell carcinoma, gastric adenocarcinoma, hepatocellular carcinoma, colonic adenocarcinoma, rectal adenocarcinoma, lung squamous cell carcinoma, bladder urothelial carcinoma, cardiac myxoma, renal clear cell carcinoma, papillary thyroid carcinoma, pancreatic carcinoma, cervical squamous cell carcinoma, cutaneous squamous cell carcinoma, non-specific invasive ductal carcinoma of breast, ovarian clear cell carcinoma, prostate carcinoma and testicular seminoma. 3 clinical specimens are contained for each type.

The human tumor tissue microarray was subjected to tissue rehydration and antigen retrieval for tissue immunofluorescence staining. The expression level of chemokine CXCL12 and the density of lymphatic vessels were detected by using anti-CXCL12 primary antibody (purchased from Bioss, Beijing) and anti-Podoplanin primary antibody (purchased from Biolegend), and observed and imaged under a laser confocal microscope (Nikon A1) using the imaging and statistic software NIS-Elements AR 3.0.

### Results

The above results showed that chemokine CXCL12 was a new pro-lymphangiogenesis factor. Therefore, in clinical, the level of chemokine CXCL12 in tumor tissues should also be associated with lymphangiogenesis. We used a human multi-tumor tissue microarray that contains totally 54 clinical specimens from 18 types of tumor tissues, including brain astrocytoma, esophageal squamous cell carcinoma, gastric adenocarcinoma, hepatocellular carcinoma, colonic adenocarcinoma, rectal adenocarcinoma, lung squamous cell carcinoma, bladder urothelial carcinoma, cardiac myxoma, renal clear cell carcinoma, papillary thyroid carcinoma, pancreatic carcinoma, cervical squamous cell carcinoma, cutaneous squamous cell carcinoma, non-specific invasive ductal carcinoma of breast, ovarian clear cell carcinoma, prostate carcinoma, and testicular seminoma. The level of chemokine CXCL12 and the density of lymphatic vessels were detected by tissue immunofluorescence, wherein the lymphatic vessels were identified by anti-human Podoplanin antibody. These clinical specimens were subsequently classified into 4 groups in accordance with the results:
high CXCL12 expression and high lymphatic vessel density
low CXCL12 expression and low lymphatic vessel density
high CXCL12 expression and low lymphatic vessel density
low CXCL12 expression and high lymphatic vessel density

The number of clinical specimens in each group was counted. The results showed that in 54 clinical specimens, the number of specimens in each group was as below:
high CXCL12 expression and high lymphatic vessel density (21/54, 38.9%)
low CXCL12 expression and low lymphatic vessel density (29/54, 53.7%)
high CXCL12 expression and low lymphatic vessel density (2/54, 3.7%)
low CXCL12 expression and high lymphatic vessel density (2/54, 3.7%)

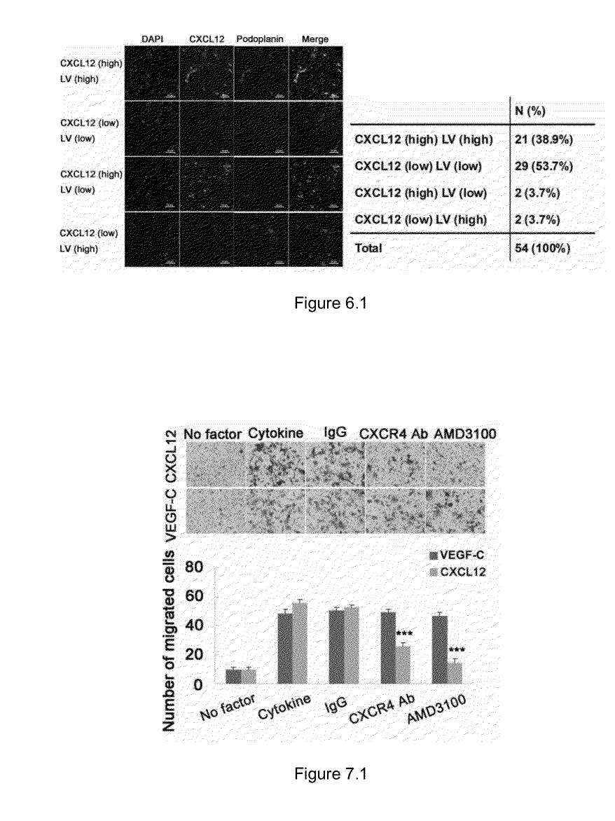

The results showed that in 54 clinical specimens, the CXCL12 level was positively correlated with the density of the newly formed lymphatic vessels in tumor tissues in more than 92% of the patients (FIG. 6.1). This result had a universal significance in a variety of tumor types, which was consistent with our in vitro and in vivo results.

### Example 7

### The Activity of Chemokine CXCL12/CXCR4 to Promote Lymphangiogenesis is Independent of the Growth Factor VEGF-C/VEGFR-3 Pathway

### Method

### 1. Effect of Antibody-Blockage of CXCR4 on the Chemotactic Activity of Chemokine CXCL12

The migration ability of mouse lymphatic endothelial cells was detected by using 8-μm-pore Transwell bucket (purchased from Costar). The bucket was placed into a 24-well plate. Mouse primary lymphatic endothelial cells (mLECs) at passage 2 or 3 in good conditions were selected and divided into 10 groups, 4 parallel samples for each group, and approximately 2×10⁴ cells for each parallel sample. The cells were digested with trypsin and then resuspended in 200 μL of fresh serum-free endothelial cell culture medium (ECM, purchased from Sciencell). The experimental grouping was as follows:
Control group without any treatment ×2;
100 ng/mL chemokine CXCL12 treatment groups;
   with isotype immunoglobulin G (IgG) control (5 μg/mL);
   with CXCR4-neutralizing antibody (5 μg/mL);
   with CXCR4 antagonist AMD3100 (25 μg/mL);
100 ng/mL growth factor VEGF-C treatment groups;
   with isotype immunoglobulin G (IgG) control (5 μg/mL);
   with CXCR4-neutralizing antibody (5 μg/mL);
   with CXCR4 antagonist AMD3100 (25 μg/mL).

The treatment groups containing CXCR4-neutralizing antibody, isotype immunoglobulin or AMD3100 were all pretreated for 30 minutes. The pretreatment process was as follows: the cells were incubated with CXCR4-neutralizing antibody (5 μg/mL, purchased from Bioss, Beijing), isotype immunoglobulin (IgG) control (5 μg/mL, prepared in the laboratory) and CXCR4 antagonist AMD3100 (25 μg/Ml, purchased from Sigma-Aldrich) for 30 minutes, respectively, and then seeded into the inner chambers of the Transwell bucket. 800 μL of serum-free endothelial cell culture medium ECM was added into each outer chamber and the corresponding agents were added to the culture medium in the outer chambers according to the experimental protocol. The culture plate was placed into an incubator and normally incubated under 5% CO₂ at 37° C. for 6 hours, followed by staining and counting.

### 2. Effect of Antibody-Blockage of VEGFR-3 on the Chemotactic Activity of Chemokine CXCL12

The migration ability of mouse lymphatic endothelial cells was detected by using 8-μm-pore Transwell bucket (purchased from Costar). The bucket was placed into a 24-well plate. Mouse primary lymphatic endothelial cells (mLECs) at passage 2 or 3 in good conditions were selected and divided into 7 groups, 4 parallel samples for each group, and approximately 2×10⁴ cells for each parallel sample. The cells were digested with trypsin and resuspended in 200 μL of fresh serum-free endothelial cell culture medium (ECM, purchased from Sciencell). The experimental grouping was as follows:
Control group without any treatment;
100 ng/mL chemokine CXCL12 treatment groups;
   with isotype immunoglobulin G (IgG) control (5 μg/mL);
   with VEGFR-3-neutralizing antibody (5 μg/mL);
100 ng/mL growth factor VEGF-C treatment groups;
   with isotype immunoglobulin G (IgG) control (5 μg/mL);
   with VEGFR-3-neutralizing antibody (5 μg/mL).

The groups containing VEGFR-3-neutralizing antibody and isotype immunoglobulin were pretreated for 30 minutes. The pretreatment process was as follows: the cells were incubated with VEGFR-3-neutralizing antibody (5 μg/mL, purchased from Bioss, Beijing), isotype immunoglobulin (IgG) control (5 ng/mL, prepared in the laboratory) for 30 minutes, respectively, and then seeded into the inner chambers of the Transwell bucket. 800 μL of fresh serum-free endothelial cell culture medium ECM was added into each outer chamber and the corresponding agents were added into the culture medium in the outer chambers according to the experimental protocol. The culture plate was placed into an incubator and normally incubated under 5% CO₂ at 37° C. for 6 hours, followed by staining and counting.

### 3. Verification of the Effect of VEGFR-3 Pathway on Chemokine CXCL12 by Matrigel Plug Assay

In the Matrigel plug assay, BABL/c mice (5 weeks old, female, purchased from Vital River Laboratories, Beijing) were prepared, totally 12 groups, 5 mice for each group. The experimental grouping was as follows:
PBS control groups ×2;
500 ng/mL chemokine CXCL12 (purchased from R&D Systems) groups;
   with isotype immunoglobulin G (IgG) control (10 ng/mL);
   with CXCR4-neutralizing antibody (10 ng/mL);
   with VEGFR-3-neutralizing antibody (10 ng/mL);
   with CXCR4 antagonist AMD3100 (50 ng/mL)
500 ng/mL growth factor VEGF-C (purchased from R&D Systems) groups;
   with isotype immunoglobulin G (IgG) control (10 ng/mL);
   with CXCR4-neutralizing antibody (10 ng/mL);
   with VEGFR-3-neutralizing antibody (10 ng/mL);
   with CXCR4 antagonist AMD3100 (50 ng/mL).

The growth factor-free Matrigel (9-10 mg/mL, purchased from Becton-Dickinson Biosciences) evenly mixed with the corresponding agents according to the experimental protocol was subcutaneously injected into the BABL/c mice along the peritoneal midline. The Matrigel formed a solid plug in the mice, and the agent was slowly released from the Matrigel to stimulate new mouse lymphatic vessels to be formed and grown into the Matrigel. 8 days later, the Matrigel was removed carefully.

The newly formed lymphatic vessels in the Matrigel were detected by immunofluorescence, and observed and imaged under a laser confocal microscope (Nikon A1) using the imaging and statistic software NIS-Elements AR 3.0.

### Results

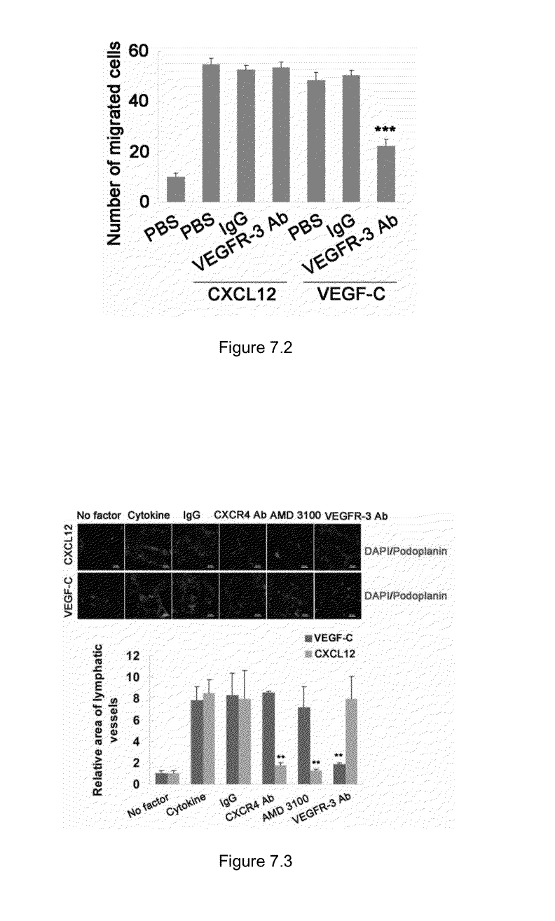

The above results demonstrated that chemokine CXCL12 was a new pro-lymphangiogenesis factor, which could recruit lymphatic endothelial cells. Whether chemokine CXCL12 exerts its functions directly via chemokine receptor CXCR4 or indirectly via other pathways? First of all, the in vitro cell chemotaxis assay demonstrated that CXCR4 pathway could mediate the recruitment of mouse lymphatic endothelial cells by chemokine CXCL12. Chemokine CXCR4-neutralizing antibody or antagonist AMD3100 could inhibit the migration of mouse lymphatic endothelial cells induced by CXCL12, but had no effect on the activity of growth factor VEGF-C (FIG. 7.1).

Among the reported pro-lymphangiogenesis factors, growth factor VEGF-C/D are the most specific and important pro-lymphangiogenesis factors which play their roles by binding to their receptor VEGFR-3. Moreover, VEGFR-3 can also mediate the functions of other pro-lymphangiogenesis factors such as basic fibroblast growth factor (bFGF) and hepatocyte growth factor (HGF). Then, can chemokine CXCL12 exerts its functions indirectly via VEGFR-3 pathway as well? Herein, we tested whether blocking VEGFR-3 pathway could affect the activity of chemokine CXCL12. In the chemotaxis assay, mouse lymphatic endothelial cells were treated with VEGFR3-neutralizing antibody at the same time. The activity of VEGF-C was significantly inhibited, but the recruitment of mouse endothelial cells by chemokine CXCL12 was not affected (FIG. 7.2).

To further confirm this result, we conducted a Matrigel plug assay in vivo. The detection of the density of lymphatic vessels in Matrigel by immunofluorescence obtained a similar result to the in vitro chemotaxis assay, i.e., VEGFR-3-neutralizing antibody did not inhibit lymphangiogenesis induced by chemokine CXCL12, either, whereas CXCR4-neutralizing antibody or antagonist AMD3100 could significantly reduce the activity of chemokine CXCL12 (FIG. 7.3). The above results demonstrated that the activity of chemokine CXCL12 to promote lymphangiogenesis was independent of VEGF-C pathway, but chemokine CXCL12 directly acted on lymphatic endothelial cells via chemokine receptor CXCR4.

### Example 8

### CXCL12 and VEGF-C have Additive Effects in Promoting Lymphangiogenesis

### Methods

### 1. Detection of Combination Effects of CXCL12 and VEGF-C by a Cell Chemotaxis Assay

The migration ability of mouse lymphatic endothelial cells was detected by using 8-μm-pore Transwell bucket (purchased from Costar). The bucket was placed into a 24-well plate. Mouse primary lymphatic endothelial cells (mLECs) at passage 2 or 3 in good conditions were selected and divided into 4 groups, 4 parallel samples for each group, and approximately 2×10⁴ cells for each parallel sample. The cells were digested with trypsin, resuspended in 200 μL of fresh serum-free endothelial cell culture medium (ECM, purchased from Sciencell), and then seeded into the inner chambers of the Transwell bucket. To each outer chamber was added 800 μL of serum-free endothelial cell culture medium mixed with 100 ng/mL of chemokine CXCL12 (purchase from R&D Systems), 100 ng/mL of VEGF-C (purchase from R&D Systems), or both CXCL12 and VEGF-C, or PBS control. The culture plate was placed into an incubator and normally incubated under 5% CO₂ at 37° C. for 6 hours, followed by staining and counting.

### 2. Detection of Combination Effects of CXCL12 and VEGF-C by a Matrigel Plug Assay

In a Matrigel plug assay, BABL/c mice (5 weeks, female, purchased from Vital River Laboratories, Beijing) were prepared, totally 12 groups, 5 mice for each group. The experimental grouping was as follows:
PBS control groups ×2;
Group with 500 ng/mL of CXCL12 (purchased from R&D Systems);
Group with 500 ng/mL of VEGF-C (purchased from R&D Systems);
Group with both 500 ng/mL of CXCL12 and 500 ng/mL of VEGF-C.

According to the experimental protocol, the growth factor-free Matrigel (9-10 mg/mL, purchased from Becton-Dickinson Biosciences) evenly mixed with the corresponding agents was subcutaneously injected into BABL/c mice along the peritoneal midline. The Matrigel formed a plug in the mice, and the agent was slowly released from the Matrigel to stimulate new lymphatic vessel to be formed and grown into the Matrigel. 8 days later, the Matrigel was removed carefully.

The newly formed lymphatic vessels in the Matrigel were detected by immunofluorescence, and observed and imaged under a laser confocal microscopy (Nikon A1) using the imaging and statistic software NIS-Elements AR 3.0.

### Results

Since CXCL12 is a new pro-lymphangiogenesis factor, and the clinical results also indicated a positive relationship between the expression level of chemokine CXCL12 and the density of newly formed tumor lymphatic vessels (FIG. 6.1), it was suggested that chemokine CXCL12 might be a good target for inhibiting tumor lymphangiogenesis and lymphatic metastasis. Considering that chemokine CXCL12 and growth factor VEGF-C are two independent pro-lymphangiogenesis factors, it is possible that they play different roles, that is, tumor tissue secretes growth factor VEGF-C to activate normal lymphatic epithelial cells, while chemokine CXCL12 abundantly present in tumor tissues can recruit the activated lymphatic epithelial cells and promote their migration to tumor tissues. Therefore, we inferred that CXCL12 and VEGF-C had additive effects in promoting lymphangiogenesis.

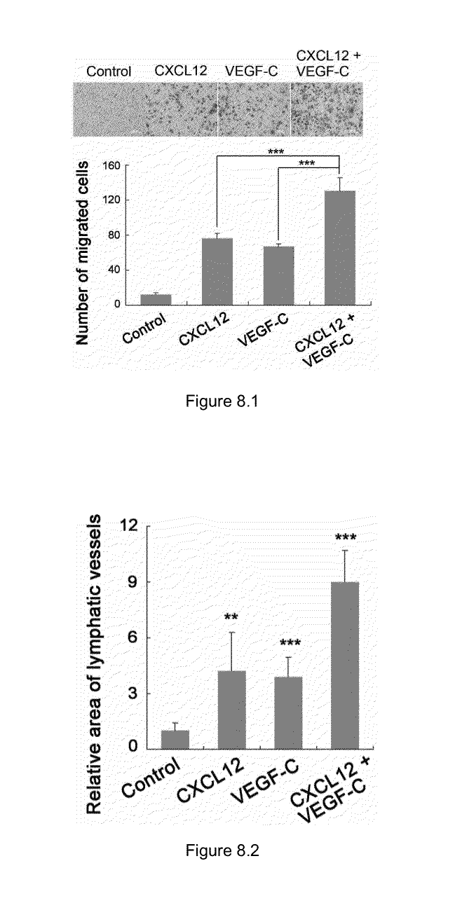

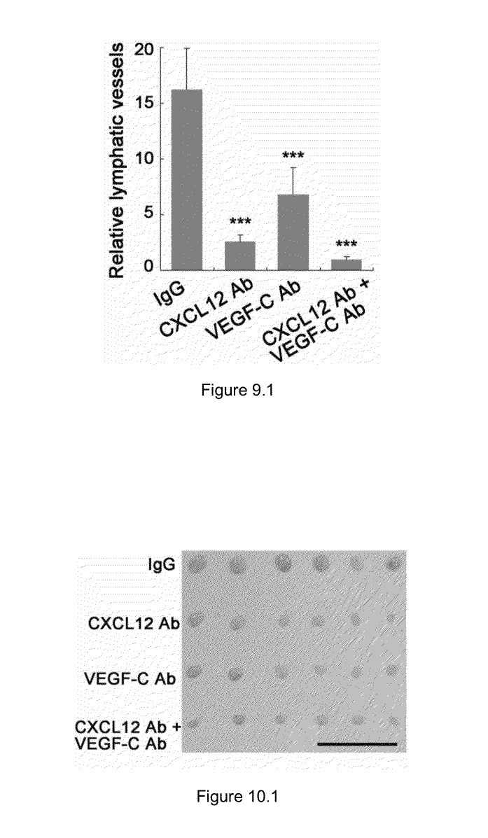

To confirm this hypothesis, we firstly proved that the concurrence of chemokine CXCL12 and growth factor VEGF-C had additive effects or synergistic effects in vitro. The results of the cell chemotaxis assay verified that chemokine CXCL12 or growth factor VEGF-C alone could promote the migration of mouse lymphatic endothelial cells; while the combined treatment with chemokine CXCL12 and growth factor VEGF-C had a better effect, which was about 2 times as good as the effect of a single agent (FIG. 8.1). In the in vivo Matrigel plug assay for lymphangiogenesis, one or both of CXCL12 and VEGF-C were mixed with Matrigel, and the lymphangiogenesis in the Matrigel was detected. In agreement with the results of in vitro cell migration assay, the combined use of chemokine CXCL12 and growth factor VEGF-C could promote lymphangiogenesis more obviously (FIG. 8.2).

### Example 9

### Blocking Both Chemokine CXCL12 and Growth Factor VEGF-C can Inhibit Lymphangiogenesis More Effectively

### Methods

### 1. Human Breast Carcinoma In Situ Nude Mouse Model

The human breast carcinoma cell line was MDA-MB-231 (purchased from American Type Culture Collection, ATCC). A stable enhanced green fluorescent protein-labeled MDA-MB-231 cell line (MDA-MB-231/eGFP) was constructed using an Enhanced Green Fluorescent Protein (eGFP) Lentivirus Kit (purchased from Genepharma, Shanghai) according to the instructions in the kit.

Taking a 24-well plate as an example, mouse lymphatic endothelial cells at passage 2 or 3 in good condition were selected. 5×10⁴ cells and 0.5 mL of normal ECM containing fetal calf serum were added to each well. The cells were incubated in an incubator under 5% CO₂ at 37° C. overnight. 3-5 gradients of virus dilutions was prepared by diluting 10 μL of lentivirus, the titer of which had been determined in advance, in ECM containing 10% fetal calf serum and polybrene (which can effectively increase transfection efficiency) in a final concentration of 5 μm/mL by 10 folds. The overnight culture solution was removed, 0.5 mL of the prepared virus dilution was added, and the culture was incubated in an incubator at 37° C. under 5% CO₂ for 8-12 hours, followed by observation of the cell condition. If there was no significant difference compared with the control group, it indicated that the toxicity was low and the culture media was not needed to be changed. After incubation for another 24 hours, the culture media was replaced with 1 mL of normal ECM containing fetal calf serum. The plate was placed in an incubator and incubated at 37° C., under 5% CO₂. Since primary cells were used, GFP fluorescence could be observed 4 days after transfection, and finally a stable MDA-MB-231/eGFP cell line could be obtained after continuous culturing for more than one week with timely culture medium replacement and passaging to ensure the cells in a good condition. Healthy nude mice, female, 6-8 weeks (purchased from Vital River Laboratories, Beijing) were prepared. The MDA-MB-231/eGFP cell line was harvested and mixed evenly with Matrigel (purchased from Becton-Dickinson Biosciences) in an equal proportion. 100 μL of suspension containing 3×10⁶ cells was inoculated subcutaneously into the mammary fad pat adjacent to inguen in each mouse. The mice were divided into 4 groups, 6 mice in each group. The experimental grouping was as follows:
Isotype IgG control group (2 mg/kg);
Group with CXCL12-neutralizing antibody (2 mg/kg);
Group with VEGF-C-neutralizing antibody (2 mg/kg);
Group with both CXCL12-neutralizing antibody (1 mg/kg) and VEGF-C-neutralizing antibody (1 mg/kg).

In accordance with the experimental grouping, the corresponding agents were injected intraperitoneally into the nude mice every day. After 3 weeks, the tumor tissues and the peritumoral inguinal lymph nodes were removed and photographed.

The removed tumor and lymph node tissues were subjected to fixing and embedding, tissue rehydration and antigen retrieval.

Tissue immunofluorescence staining: The tumor tissue sections subjected to antigen retrieval were stained with anti-podoplanin primary antibody (purchased from Santa Cruz Biotechnology) by tissue immunofluorescence. The sections were observed and imaged under a laser confocal microscope (Nikon A1) using the imaging and statistic software NIS-Elements AR 3.0.

### Results

Since chemokine CXCL12 and growth factor VEGF-C utilize two independent mechanisms of action, both are involved in the regulation of lymphangiogenesis, and have additive effects in promoting lymphangiogenesis when used in combination, we tried to block both chemokine CXCL12 and growth factor VEGF-C with antibodies, in attempt to effectively inhibit tumor lymphangiogenesis, thereby treating tumor metastasis. Therefore, we constructed a human breast carcinoma in situ nude mouse model to study the effect of combined blockage of chemokine CXCL12 and growth factor VEGF-C in controlling tumor lymphangiogenesis and lymphatic metastasis. Firstly, lentivirus was utilized to construct a stable enhanced green fluorescent protein (eGFP)-labeled MDA-MB-231 cell line (MDA-MB-231/eGFP), which can be used to observe the metastasis of breast cancer cells in vivo. After the tumor was inoculated into the mammary fad pat of the nude mice, CXCL12-neutralizing antibody and VEGF-C-neutralizing antibody were injected intraperitoneally into the mice. Then tumor tissues were isolated from the mice to determine the density of tumor lymphatic vessels by tissue immunofluorescence. The statistical results of laser confocal microscopy showed that CXCL12-neutralizing antibody remarkably reduced the density of newly formed lymphatic vessels in the breast tumor tissues, and blocking both chemokine CXCL12 and growth factor VEGF-C could inhibit lymphangiogenesis more effectively than each alone.

### Example 10

### Blocking Both Chemokine CXCL12 and Growth Factor VEGF-C can Inhibit Tumor Lymphatic Metastasis More Effectively

### Methods

Lymph node tissue sections from the human breast carcinoma in situ nude mouse model could be used to directly observe enhanced green fluorescent protein-labeled MDA-MB-231 breast cancer cells which metastasized to the lymph nodes. Without immunofluorescence staining, the nuclei were directly stained by DAPI, and then rinsed with PBS for 5 times, 5 minutes each time. The sections were mounted with Clearmount (Beijing Zhongshan Golden Bridge Corporation), and observed and imaged under a laser confocal microscope (Nikon A1) using the imaging and statistic software NIS-Elements AR 3.0.

### Results

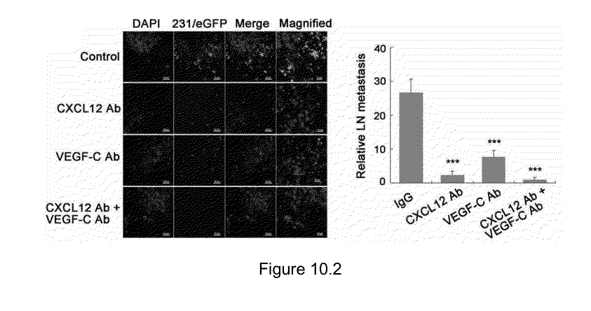

In the human breast carcinoma in situ nude mouse model, peritumoral inguinal lymph nodes were removed from tumor-bearing mice to analyze the metastasis of breast carcinoma lymph nodes. The peritumoral lymph nodes of the mice were observed, and it was found that the swelling of the inguinal lymph nodes of the mice in the antibody-treated groups was much better than that of the mice in the isotype immunoglobulin (IgG) control group (FIG. 10.1).

Since the breast cancer cell line was labeled with green fluorescent protein, the metastasized tumor cells in the lymph nodes could be observed directly under a laser confocal microscope. Further observation showed that there were almost no metastasized breast cancer cells in the lymph nodes of the mice in the groups where both chemokine CXCL12 and growth factor VEGF-C were blocked (FIG. 10.2). This result verified our hypothesis: on one hand, blocking CXCL12 can inhibit lymphatic metastasis of breast cancer cells; on the other hand, a multi-target combination treatment with antibodies blocking both chemokine CXCL12 and growth factor VEGF-C pathways can control tumor lymphatic metastasis more effectively.

### REFERENCES

1. Alitalo K, Tammela T, Petrova T V. Lymphangiogenesis in development and human disease. Nature, 2005, 438: 946-953.
2. Wilting J, Papoutsi M, Christ B, et al. The transcription factor Prox1 is a marker for lymphatic endothelial cells in normal and diseased human tissues. FASEB J, 2002, 16: 1271-1273.
3. Breiteneder-Geleff S, Soleiman A, Kowalski H, et al. Angiosarcomas express mixed endothelial phenotypes of blood and lymphatic capillaries: podoplanin as a specific marker for lymphatic endothelium. Am J Pathol, 1999, 154: 385-394.
4. Prevo R, Banerji S, Ferguson D J, et al. Mouse LYVE-1 is an endocytic receptor for hyaluronan in lymphatic endothelium. J Biol Chem, 2001, 276: 19420-19430.
5. Partanen T A, Arola J, Saaristo A, et al. VEGF-C and VEGF-D expression in neuroendocrine cells and their receptor, VEGFR-3, in fenestrated blood vessels in human tissues. FASEB J, 2000, 14: 2087-2096.
6. Cueni L N, Detmar M. The lymphatic system in health and disease. Lymphat Res Biol, 2008, 6: 109-122.
7. Saharinen P, Tammela T, Karkkainen M J, et al. Lymphatic vasculature: development, molecular regulation and role in tumor metastasis and inflammation. Trends Immunol, 2004, 25: 387-395.
8. Tobler N E, Detmar M. Tumor and lymph node lymphangiogenesis—impact on cancer metastasis. J Leukoc Biol, 2006, 80: 691-696.
9. Skobe M, Hawighorst T, Jackson D G, et al. Induction of tumor lymphangiogenesis by VEGF-C promotes breast cancer metastasis. Nat Med, 2001, 7: 192-198.
10. Thiele W, Sleeman J P. Tumor-induced lymphangiogenesis: a target for cancer therapy? J Biotechnol, 2006, 124: 224-241.
11. Joukov V, Pajusola K, Kaipainen A, et al. A novel vascular endothelial growth factor, VEGF-C, is a ligand for the Flt4 (VEGFR-3) and KDR (VEGFR-2) receptor tyrosine kinases. EMBO J, 1996, 15: 290-298.
12. Achen M G, Roufail S, Domagala T, et al. Monoclonal antibodies to vascular endothelial growth factor-D block its interactions with both VEGF receptor-2 and VEGF receptor-3. Eur J Biochem, 2000, 267: 2505-2515.
13. Makinen T, Veikkola T, Mustjoki S, et al. Isolated lymphatic endothelial cells transduce growth, survival and migratory signals via the VEGF-C/D receptor VEGFR-3. EMBO J, 2001, 20: 4762-4773.
14. Veikkola T, Jussila L, Makinen T, et al. Signalling via vascular endothelial growth factor receptor-3 is sufficient for lymphangiogenesis in transgenic mice. EMBO J, 2001, 20: 1223-1231.
15. Karkkainen M J, Ferrell R E, Lawrence E C, et al. Missense mutations interfere with VEGFR-3 signalling in primary lymphoedema. Nat Genet, 2000, 25: 153-159.
16. Enholm B, Karpanen T, Jeltsch M, et al. Adenoviral expression of vascular endothelial growth factor-C induces lymphangiogenesis in the skin. Circ Res, 2001, 88: 623-629.
17. Rissanen T T, Markkanen J E, Gruchala M, et al. VEGF-D is the strongest angiogenic and lymphangiogenic effector among VEGFs delivered into skeletal muscle via adenoviruses. Circ Res, 2003, 92: 1098-1106.
18. Cao Y, Linden P, Farnebo J, et al. Vascular endothelial growth factor C induces angiogenesis in vivo. Proc Natl Acad Sci USA, 1998, 95: 14389-14394.
19. Byzova T V, Goldman C K, Jankau J, et al. Adenovirus encoding vascular endothelial growth factor-D induces tissue-specific vascular patterns in vivo. Blood, 2002, 99: 4434-4442.
20. Cueni L N, Detmar M. New insights into the molecular control of the lymphatic vascular system and its role in disease. J Invest Dermatol, 2006, 126: 2167-2177.
21. Tammela T, Saaristo A, Lohela M, et al. Angiopoietin-1 promotes lymphatic sprouting and hyperplasia. Blood, 2005, 105: 4642-4648.
22. Gale N W, Thurston G, Hackett S F, et al. Angiopoietin-2 is required for postnatal angiogenesis and lymphatic patterning, and only the latter role is rescued by Angiopoietin-1. Dev Cell, 2002, 3: 411-423.
23. Kajiya K, Hirakawa S, Ma B, et al. Hepatocyte growth factor promotes lymphatic vessel formation and function. EMBO J, 2005, 24: 2885-2895.
24. Kubo H, Cao R, Brakenhielm E, et al. Blockade of vascular endothelial growth factor receptor-3 signaling inhibits fibroblast growth factor-2-induced lymphangiogenesis in mouse cornea. Proc Natl Acad Sci USA, 2002, 99: 8868-8873.
25. Cao R, Bjorndahl M A, Religa P, et al. PDGF-BB induces intratumoral lymphangiogenesis and promotes lymphatic metastasis. Cancer Cell, 2004, 6: 333-345.
26. Bjorndahl M, Cao R, Nissen L J, et al. Insulin-like growth factors 1 and 2 induce lymphangiogenesis in vivo. Proc Natl Acad Sci USA, 2005, 102: 15593-15598.
27. Murphy P M, Baggiolini M, Charo I F, et al. International union of pharmacology. XXII. Nomenclature for chemokine receptors. Pharmacol Rev, 2000, 52: 145-176.
28. Baggiolini M. Chemokines and leukocyte traffic. Nature, 1998, 392: 565-568.
29. Moser B, Loetscher P. Lymphocyte traffic control by chemokines. Nat Immunol, 2001, 2: 123-128.
30. Bleul C C, Farzan M, Choe H, et al. The lymphocyte chemoattractant SDF-1 is a ligand for LESTR/fusin and blocks HIV-1 entry. Nature, 1996, 382: 829-833.
31. Fredriksson R, Lagerstrom M C, Lundin L G, et al. The G-protein-coupled receptors in the human genome form five main families. Phylogenetic analysis, paralogon groups, and fingerprints. Mol Pharmacol, 2003, 63: 1256-1272.
32. Feng Y, Broder C C, Kennedy P E, et al. HIV-1 entry cofactor: functional cDNA cloning of a seven-transmembrane, G protein-coupled receptor. Science, 1996, 272: 872-877.
33. Balkwill F. The significance of cancer cell expression of the chemokine receptor CXCR4. Semin Cancer Biol, 2004, 14: 171-179.
34. Smith M C, Luker K E, Garbow J R, et al. CXCR4 regulates growth of both primary and metastatic breast cancer. Cancer Res, 2004, 64: 8604-8612.
35. Phillips R J, Burdick M D, Lutz M, et al. The stromal derived factor-1/CXCL12-CXC chemokine receptor 4 biological axis in non-small cell lung cancer metastases. Am J Respir Crit. Care Med, 2003, 167: 1676-1686.
36. Scotton C J, Wilson J L, Scott K, et al. Multiple actions of the chemokine CXCL12 on epithelial tumor cells in human ovarian cancer. Cancer Res, 2002, 62: 5930-5938.
37. Pan J, Mestas J, Burdick M D, et al. Stromal derived factor-1 (SDF-1/CXCL12) and CXCR4 in renal cell carcinoma metastasis. Mol Cancer, 2006, 5: 56.
38. Taichman R S, Cooper C, Keller E T, et al. Use of the stromal cell-derived factor-1/CXCR4 pathway in prostate cancer metastasis to bone. Cancer Res, 2002, 62: 1832-1837.
39. Geminder H, Sagi-Assif O, Goldberg L, et al. A possible role for CXCR4 and its ligand, the CXC chemokine stromal cell-derived factor-1, in the development of bone marrow metastases in neuroblastoma. J Immunol, 2001, 167: 4747-4757.
40. Schioppa T, Uranchimeg B, Saccani A, et al. Regulation of the chemokine receptor CXCR4 by hypoxia. J Exp Med, 2003, 198: 1391-1402.
41. Staller P, Sulitkova J, Lisztwan J, et al. Chemokine receptor CXCR4 downregulated by von Hippel-Lindau tumour suppressor pVHL. Nature, 2003, 425: 307-311.
42. Chu C Y, Cha S T, Chang C C, et al. Involvement of matrix metalloproteinase-13 in stromal-cell-derived factor 1 alpha-directed invasion of human basal cell carcinoma cells. Oncogene, 2007, 26: 2491-2501.
43. Iruela-Arispe M L, Bornstein P, Sage H. Thrombospondin exerts an antiangiogenic effect on cord formation by endothelial cells in vitro. Proc Natl Acad Sci USA, 1991, 88: 5026-5030.
44. Hirakawa S, Hong Y K, Harvey N, et al. Identification of vascular lineage-specific genes by transcriptional profiling of isolated blood vascular and lymphatic endothelial cells. Am J Pathol, 2003, 162: 575-586.
45. Hu X, Dai S, Wu W J, et al. Stromal cell derived factor-1 alpha confers protection against myocardial ischemia/reperfusion injury: role of the cardiac stromal cell derived factor-1 alpha CXCR4 axis. Circulation, 2007, 116: 654-663.
46. Singh S, Srivastava S K, Bhardwaj A, et al. CXCL12-CXCR4 signalling axis confers gemcitabine resistance to pancreatic cancer cells: a novel target for therapy. Br J Cancer, 103: 1671-1679.

1. Use of a CXCR4 inhibitor and/or a CXCL12 inhibitor in the manufacture of a preparation for inhibiting lymphangiogenesis in a subject.

2. The use according to claim 1, wherein said CXCR4 inhibitor is selected from the group consisting of an anti-CXCR4 antibody or an active fragment thereof, and CXCR4 antagonist AMD3100; and
said CXCL12 inhibitor is selected from the group consisting of an anti-CXCL12 antibody, a CXCL12 antagonist, and a soluble CXCR4 fragment which competitively binds to CXCL12.

3. The use according to claim 1 or 2, wherein said subject suffers from a cancer, inflammation and/or transplant rejection.

4. Use of a CXCR4 inhibitor and/or a CXCL12 inhibitor in the preparation of a medicament for inhibiting tumor lymphatic metastasis in a cancer patient.

5. The use according to claim 4, wherein said CXCR4 inhibitor is selected from the group consisting of an anti-CXCR4 antibody or an active fragment thereof, and CXCR4 antagonist AMD3100; and
said CXCL12 inhibitor is selected from the group consisting of an anti-CXCL12 antibody, a CXCL12 antagonist, and a soluble CXCR4 fragment which competitively binds to CXCL12.

6. Use of (a) a CXCR4 inhibitor and/or a CXCL12 inhibitor, and (b) a VEGF-C inhibitor and/or a VEGF-D inhibitor and/or a VEGFR-3 inhibitor, in the preparation of a medicament for inhibiting tumor lymphatic metastasis in a cancer patient.

7. The use according to claim 6, wherein said CXCR4 inhibitor is selected from the group consisting of an anti-CXCR4 antibody or an active fragment thereof, and CXCR4 antagonist AMD3100;
said CXCL12 inhibitor is selected from the group consisting of an anti-CXCL12 antibody, a CXCL12 antagonist, and a soluble CXCR4 fragment which competitively binds to CXCL12;
said VEGF-C inhibitor is selected from the group consisting of an anti-VEGF-C antibody, a VEGF-C antagonist, and a soluble fragment of VEGFR-3 or VEGFR-2 which competitively binds to VEGF-C;
said VEGF-D inhibitor is selected from the group consisting of an anti-VEGF-D antibody, a VEGF-D antagonist, and a soluble fragment of VEGFR-3 or VEGFR-2 which competitively binds to VEGF-D; and
said VEGFR-3 inhibitor is selected from the group consisting of an anti-VEGFR-3 antibody and an antagonist which inhibits the activity of VEGFR-3 tyrosine kinase.

8. A pharmaceutical composition for inhibiting tumor lymphatic metastasis in a cancer patient, comprising:
(a) a CXCR4 inhibitor and/or a CXCL12 inhibitor, and (b) a VEGF-C inhibitor and/or a VEGF-D inhibitor and/or a VEGFR-3 inhibitor, as active ingredients; and
optionally a pharmaceutically acceptable carrier.

9. A kit for inhibiting tumor lymphatic metastasis in a cancer patient, comprising:
(a) a CXCR4 inhibitor and/or a CXCL12 inhibitor; and
(b) a VEGF-C inhibitor and/or a VEGF-D inhibitor and/or a VEGFR-3 inhibitor.
